(19) United States
(12) Patent Application Publication
(10) Pub. No.: US 2023/0302131 A1
(43) Pub. Date: Sep. 28, 2023

(54) MPC INHIBITION FOR PRODUCING T-CELLS WITH A MEMORY PHENOTYPE

(71) Applicant: UNIVERSITÉ DE LAUSANNE, Lausanne (CH)

(72) Inventors: Pedro ROMERO, Epalinges (CH); Mathias WENES, Chexbres (CH)

(21) Appl. No.: 18/021,506

(22) PCT Filed: Aug. 23, 2021
(86) PCT No.: PCT/EP2021/073299

§ 371 Date: Feb. 15, 2023

(30) Foreign Application Priority Data
Aug. 21, 2020 (EP) 20192220.0

(51) Int. Cl. A61K 39/00; C12N 5/0783; A61K 39/39; A61P 35/00

(52) U.S. Cl. CPC A61K 39/4611; C12N 5/0636; A61K 39/39; A61P 35/00; C12N 2501/999; C12N 2501/2302; C12N 2501/2307; A61K 2039/55511; A61K 2239/39

(57) ABSTRACT

The present invention relates to an in vitro cell culture method comprising a step of contacting T-cells with an MPC inhibitor, and further to a cell population comprising T-cells with a memory phenotype obtained by said method, preferably, wherein the T-cells are human cells. The present invention also relates to a method for generating and/or maintaining T-cells and/or B-cells with a memory phenotype comprising the steps of culturing T-cells and or B-cells in vitro and adding an MPC inhibitor to the culture. The invention furthermore relates to a population of T-cells and/or B-cells obtained by the methods of the invention. Also provided are immunotherapies using the cells of the invention. Furthermore, provided is an MPC inhibitor for use in immunotherapy and/or as a vaccine co-adjuvant.

The present invention relates to an in vitro cell culture method comprising a step of contacting activated T-cells with an MPC inhibitor, and further to a cell population comprising T-cells with a memory phenotype obtained by said method, preferably, wherein the T-cells are human cells. The present invention also relates to a method for generating and/or maintaining T-cells with a memory phenotype comprising the steps of culturing T-cells in vitro and adding an MPC inhibitor to the culture. The invention furthermore relates to a population of T-cells obtained by the methods of the invention. Also provided are immunotherapies using the cells of the invention. Furthermore, provided is an MPC inhibitor for use in immunotherapy and/or as a vaccine co-adjuvant.

Adoptive cell transfer (ACT) immunotherapy is showing impressive objective tumor responses in several hematological malignancies and in advanced metastatic melanoma. However, both the magnitude of tumor responses and the fraction of patients benefitting from this novel therapeutic approach remains limited. T lymphocytes prepared for ACT are generally terminally differentiated, resulting in inefficient engraftment, limited persistence and cancer recurrence. It has been shown in mouse tumor models that the infusion of T cells with a self-renewing, memory phenotype confers a stronger and more sustained anti-tumor response. Recently, it is becoming clear that T cell fate is tightly linked with specific metabolic characteristics.

CD8+ T cells are crucial mediators of the adaptive immune response against cancer cells and pathogenic intracellular microorganisms such as viruses. Upon antigen stimulation, naïve CD8+ T cells undergo extensive clonal expansion and differentiation into effector cells. A major proportion of these cells are short-lived effector cells (SLEC) that are terminally differentiated and characterized by a potent cytotoxic potential, as well as by the ability to produce large amounts of inflammatory cytokines, such as IFN and TNF. The remaining effector cells are memory precursor effector cells (MPEC), that will further differentiate into long-lived, self-renewing memory CD8+ T cells, able to rapidly proliferate into efficient effector cells upon re-challenge (Nat Rev Immunol 8, 107-119 (2008)). This self-renewal and multipotency are capacities that characterize the ideal immune cell, fit for adoptive cell transfer (ACT) immunotherapy against cancer, and able to overcome some of the current issues with ACT (Nat Rev Cancer 12, 671-684 (2012)). Indeed, despite showing promising results in a fraction of patients, T lymphocytes prepared for ACT are generally terminally differentiated, resulting in inefficient engraftment and cancer recurrence. It has been shown in mouse tumor models that the infusion of T cells with a self-renewing, memory phenotype confers a stronger and more sustained anti-tumor response (Clin Cancer Res 17, 5343-5352 (2011)).

A multitude of molecular pathways, transcription factors and epigenetic imprinting has been shown to drive effector versus memory CD8+ T cell differentiation, leading to the differential expression of genes and surface molecules that can be used to discriminate between these subpopulations (Nat Rev Immunol 12, 749-761 (2012)). It has become clear that those processes are all closely intertwined with the cell's metabolism. Indeed, drastic changes in T cell metabolism are required to support their activation and function. The high energetic and biosynthetic requirements of clonally expanding effector cells are provided by aerobic glycolysis and TCA cycle activity, while resting, long-lived memory T cells rather rely on oxidative phosphorylation fueled by fatty acids (Nature 460, 103-107 (2009), Nat Methods 10, 1213-1218 (2013), Immunity 41, 75-88 (2014)). It has been shown that the inhibition or stimulation of certain metabolic processes alone is sufficient to induce effector versus memory differentiation, with corresponding in vivo effects when the cells are re-infused in infected or tumor-bearing mice (Immunity 44, 1312-1324 (2016), Immunity 45, 1024-1037 (2016)). However, current strategies have been shown to dramatically inhibit T cell proliferation and are therefore not suitable for use as a supplement during ex vivo T cell expansion cultures for ACT cancer immunotherapy.

Thus, there is a need for improved methods to obtain T-cells with a memory phenotype. There is also a need to expand the repertoire of compounds which can promote a memory phenotype during in vitro expansion of T-cells for the development of new immunotherapies.

The technical problem is solved by the embodiments provided herein and as characterized in the claims.

Thus, in a first embodiment, the invention relates to an in vitro cell culture method comprising a step of contacting T-cells with an inhibitor of the mitochondrial pyruvate carrier (MPC inhibitor).

It was surprisingly found by the inventors that inhibiting the mitochondrial pyruvate carrier (MPC) during CD8 T cell priming in vitro, leads to a significant increase in mitochondrial oxygen consumption. This metabolic adaptation was accompanied by an increased surface expression of the central memory marker CD62L. Adoptive transfer of MPC-inhibited CD8 T cells into melanoma tumor-bearing mice, resulted in a better tumor control compared to DMSO-treated cells. A much higher proportion of adoptively transferred treated CD8 T cells formed central memory cells. Furthermore, upon MPC inhibition, T cells infiltrating the tumor were characterized by a reduced PD-1 expression and increased cytokine production. Mechanistically, altered metabolic fluxes following MPC inhibition led to an increase in acetyl-CoA levels. This was accompanied by an elevated acetylation and methylation of histones, resulting in the modulation of epigenetic modifications promoting activation of memory gene expression. Thus, it was demonstrated that metabolic adaptations induced during ex vivo CD8 T cell expansion can lead to a long-lasting central memory T cell differentiation, thereby improving their anti-tumoral therapeutic potential upon adoptive cell transfer.

In particular herein, and in the context of the invention, when reference is made to T-cells, the T-cells preferably are or comprise CD8+ T-cells. Furthermore, the terms “CD8+ T-cells” and “cytotoxic T-cells” are used interchangeably herein.

MPC inhibition during in vitro priming of CD8 T-cells was found to induce memory marker expression and to result in increased central memory formation upon in vivo transfer in a mouse bacterial infection model; see appended Examples.

As such, the invention is, at least partly, based on the surprising discovery that T-cells, in particular CD8+ T-cells, with a memory phenotype can be obtained from in vitro culture, when an MPC inhibitor is added to the culture, or in other words, when the T-cells are contacted with an MPC inhibitor during culture, as demonstrated in the appended Examples. In particular, the obtained T-cells are enriched for T-cells with a memory phenotype. It is further a surprising discovery that said T-cells can be efficiently activated and obtained with high efficiency, despite having a memory phenotype. In addition, it was surprisingly found by the inventors that CD8+ T-cells exist at higher numbers and comprise more cells with a memory phenotype in vivo upon adoptive cell transfer when they have been cultured in the presence of an MPC inhibitor in vitro. It was also surprisingly found that cancer-specific adoptively transferred CD8+ T-cells have an enhanced anti-cancer activity in vivo when they have been cultured in the presence of an MPC inhibitor in vitro. The present invention provides thus more effective cancer-specific CD8+ T-cells, which may be used to improve commonly practiced anti-cancer T-cell immunotherapies.

The invention is, however, not limited to CD8+ T-cells, but an MPC inhibitor may be also used to obtain other T-cells, e.g. CD4+ T-cells, with a memory phenotype. Furthermore, the inventors have found that contacting T-cells during culture with an MPC inhibitor promotes the generation of T-cells with a memory phenotype with both, mouse and human cells. In particular, since the key findings with mouse T-cells could be reproduced with human cells, i.e. peripheral blood mononuclear cells (PBMC) from adults or from human umbilical cord blood (CB, CBMC), the culture methods and cell populations of the invention may be highly useful for T-cell based immunotherapies for human patients, e.g. cancer patients. Moreover, it was found that the human CD8+ T-cells with a memory phenotype that were produced by culturing human T-cells in the presence of an MPC inhibitor were more effectively restimulated than human CD8+ T-cells with a memory phenotype that were produced by culturing in the absence of an MPC inhibitor.

In a preferred embodiment, the T-cells are activated before and/or during culture, preferably during culture, in particular while the cells are contacted with the MPC inhibitor, and preferably from beginning of the culture. Activated T-cells with a memory phenotype may be preferable for adoptive cell transfer therapies to rapidly generate a large amount of antigen-specific effector cells in vivo, but without rapid exhaustion of the transferred cell pool. In vitro cell culture comprising an MPC inhibitor allows activation of naïve T-cells and/or allows maintenance of activated T-cells during in vitro culture. Moreover, as shown in the appended Examples, the T-cells, i.e. the human T-cells, generated according to the method of the invention can be effectively reactivated, and respond to a restimulation cue with an increased production of Interferon-gamma (IFNγ) compared to T-cells that were cultured in the absence of a MPC inhibitor. This suggests that the T-cells with a memory phenotype provided herein have superior properties, which may be highly advantageous for their use in immunotherapy.

In certain embodiments, IL-2, IL-7 and/or one or more antigenic peptide(s) are added to the culture. In particular, IL-2 is added during activation and the contacting with the MPC inhibitor provided herein. IL-2, IL-7 and/or one or more antigenic peptide(s) further promote the generation and/or maintenance of activated T-cells with a memory phenotype.

Accordingly, the invention further relates to a cell population comprising T-cells with a memory phenotype obtained by inventive method provided herein, preferably wherein the T-cells are human cells. Said cell population is also called a population of T-cells herein, because it is preferred that the cell population consists predominantly or exclusively of T-cells, for example, wherein at least 90%, 95%, 98% or 99% of the cells in the population are T-cells. Thus, said cell population may be a population of T-cells obtained by the method of the invention which comprises a higher proportion of T-cells with a memory phenotype and/or shows in average a more pronounced memory phenotype compared to a population of T-cells obtained in parallel by the same method except that no MPC inhibitor is added to the culture (DMSO control). Moreover, it is preferred that at least 30%, 40%, 50%, 60%, 70%, 80%, or 90%, or at least 95%, 97%, 98% or 99%, preferably at least 70%, of the cells in the inventive cell population provided herein are T-cells with a memory phenotype, in particular wherein said T-cells with a memory phenotype express CD62L.

The cell population provided herein may be used in immunotherapy as described herein, in particular wherein the cell population or the T-cells comprised in said cell population is/are administered to a patient.

Furthermore, the invention relates to a population of T-cells obtained by the method of the invention which comprises a higher proportion of T-cells with a memory phenotype and/or shows in average a more pronounced memory phenotype compared to a population of T-cells obtained in parallel by the same method except that no MPC inhibitor is added to the culture (DMSO control).

In one embodiment, the population of cells are mouse cells, and the proportion of cytotoxic T-cells showing surface expression of CD62L after about 7 days of culture is about 60 to 75%.

In one embodiment, the population of cells are mouse cells, and the mean fluorescence intensity of CD62L in cytotoxic T-cells therein is about 1.5-fold to 2-fold increased compared to a DMSO control.

Accordingly, the invention further relates to an MPC inhibitor for use in immunotherapy. In particular, the immunotherapy may comprise administering T-cells to a patient, wherein the T-cells have been contacted with the MPC inhibitor during the in vitro culture according to the inventive method provided herein. In particular, said T-cells have acquired a memory phenotype during said in vitro culture.

Accordingly, the invention further relates to a population of T-cells obtained by the method of the invention, i.e. a cell population comprising T-cells with a memory phenotype as provided herein, for use in immunotherapy.

Accordingly, the invention further relates to an immunotherapy comprising administering an MPC inhibitor and/or a population of T-cells contacted with an MPC inhibitor to a patient.

In certain embodiments, the immunotherapy comprises T-cells which acquire or have acquired a memory phenotype within a subject, in vitro and/or ex vivo.

The application of an MPC inhibitor to promote the generation and/or maintenance of T-cells with a memory phenotype is not limited to a specific time between the isolation of primary cells and the end of the cell therapy or a specific location of the cells. However, it is preferred that the T-cells are contacted with the MPC inhibitor at least during activation, preferably from the beginning of the culture and/or activation, for example for the first 3 or 4 days. Furthermore, the T-cells may be contacted with the MPC inhibitor during the entire culture period. However, it is also possible to wash the MPC inhibitor away after the initial activation phase (priming phase), and continue the culture without an MPC inhibitor, e.g. with a medium comprising IL-2 and IL-7.

In particular, the activation phase, which may also be called “the priming phase”, may take place from day 0 to day 3 or 4, i.e. for the first three or four days of the culture. During that phase, the foundations for effector vs memory differentiation are made. However, only towards the end of the culture, the T cells are fully differentiated and matured into memory T cells, for example until day 7 for mouse cells, or day 10 to 11 for human T-cells. As described herein, the MPC inhibitor is preferably present in the activation phase but may be still present during the subsequent maturation phase. At the end of the culture, the T-cells with a memory phenotype may be restimulated to determine their reactivation capacity (recall potential), as described herein.

An MPC inhibitor may be administered to T-cells and/or B-cells within a subject (in vivo), in vitro and/or ex vivo. The T-cells may be in a culture dish/flask or be within a subject, for example in the blood, lymph or tissue, for example a lymphoid organ or a tumor, when contacted with an MPC inhibitor. Preferably, the T-cells are contacted with the MPC inhibitor in vitro during culture.

In a preferred embodiment, the immunotherapy is a T-cell therapy. Preferably, the T-cell therapy comprises CD8+(cytotoxic) T-cells. Cytotoxic T-cells with a memory phenotype obtained upon contact with an MPC inhibitor may persist for a prolonged period of time within a subject, for example, upon adoptive cell transfer. They may also produce a larger number of cytotoxic effector T-cells for a longer time which may cause efficient lysis of the target cells. A sustained cytotoxic activity of transferred CD8+ T-cells with a memory phenotype may lead to a durable depletion, and in some cases even permanent elimination, of the target cells, for example, cancer cells.

In certain embodiments, the immunotherapy is a therapy to treat cancer, a chronic viral infection or an autoimmune disease.

Cytotoxic T-cells with a memory phenotype are particularly suitable to eliminate cells, i.e. mediate the elimination of cells, for example cancer cells or cells infected by a virus. Cancer cells or cells infected by virus are preferred targets, at least partly because they may express specific neoantigens or virus-derived antigens which may reduce the risk of non-specifically targeting healthy cells. Certain subsets of CD4+ T-cells (helper T-cells) with a memory phenotype may also contribute to the elimination of target cells, either directly by generating cells with a cell-lytic activity or indirectly by stimulating/activating CD8+ T-cells. CD4+ regulatory T-cells with a memory phenotype, in contrast, may promote an immunosuppressive environment, for example in certain tissues and/or in proximity of certain T-cells. Such an immunosuppressive activity may be particularly desired for the treatment of autoimmune diseases.

In a preferred embodiment, the immunotherapy is a therapy to treat cancer. T-cells with a memory phenotype may be also effective in the treatment of aggressive and/or late-stage cancer.

In a preferred embodiment, the cancer is resistant to chemotherapy, targeted therapy and/or antibody-mediated immunotherapy and/or comprises metastases.

In particular herein, e.g. in certain embodiments, the immunotherapy may comprise transfer of in vitro or ex vivo cultured T-cells into a subject. In particular, T-cells and/or B-cells are obtained from a subject, preferably the patient (autologous cells), cultured in vitro (ex vivo) and adoptively transferred into the patient.

In a preferred embodiment of the invention, the cells are T-cells. In a very preferred embodiment, the T-cells comprise cytotoxic T-cells (CD8+ T-cells).

In certain embodiments, the memory phenotype comprises higher expression of CD62L, TCF1, CD127, CCR7, CD27 and/or CD28, lower expression of KLRG1, and/or an increased basal oxygen consumption, maximal respiratory capacity and/or spare respiratory capacity compared to a control. In particular, in the context of the present invention, the control refers to a control treatment of T-cells and/or B-cells which does not comprise an MPC inhibitor and is preferably a solvent control, for example comprising DMSO. Preferably, the control treatment is performed in parallel with a comparable population of cells and, where necessary, comparable subjects, and all steps are identical except that the respective solvent is used instead of the MPC inhibitor. When the protein is expressed at the cell surface, it is preferably detected at the cell surface. An increased basal oxygen consumption, maximal respiratory capacity and/or spare respiratory capacity is indicative of a mitochondrial metabolism, at least partially through fatty acid oxidation which is associated, as known in the art, with a memory phenotype rather than an effector phenotype (Zhang (2018) Trends Mol Med 24(1):30-48) Preferably, the memory phenotype comprises higher expression of CD62L, preferably at the cell surface.

In certain embodiments, the T-cells comprise T-cells derived, i.e differentiated, from tumor-infiltrating lymphocytes (TILs). In particular, the TILs are isolated from a tumor of the same patient which receives the TILs, i.e. the in vitro expanded TILs, upon MPC treatment during in vitro culture. Preferably, the TILs are T-cells. TILs are enriched for T-cells specific for the cancer of a patient and therefore a suitable basis for cancer T-cell therapy, i.e. a suitable starting cell population for the inventive culture method provided herein. As known in the art, however, TILs comprise mainly, but not necessarily only, effector and/or senescent cells. MPC treatment may thus be useful for enriching TILs with a memory phenotype.

In certain embodiments, the T-cells comprise a heterologous antigen receptor, preferably a T-cell receptor (TCR) or a chimeric antigen receptor (CAR). There are methods known in the art, to identify and select a TCR or generate a CAR which is specific for a certain antigen, see e.g. Weber (2020), Cell 181(1):46-62, and June (2018), N Engl J Med 379(1):64-73. Establishing antigen-specificity by genetic engineering such that the T-cells express the desired TCR or CAR may increase the repertoire of suitable T-cells for in vitro culture and subsequent adoptive cell transfer. In particular, naïve T-cells (which are not yet specific for antigen) can be isolated from a patient or MHC/HLA matched subject, genetically manipulated to express a certain TCR or CAR, activated and differentiated into T-cells with a memory phenotype.

In particular herein, in a preferred embodiment of the invention, the MPC inhibitor comprises one or more small molecule(s). In a very preferred embodiment, the small molecule(s) is/are UK5099, Pioglitazone, Rosiglitazone, MSDC-0602, MSDC-0160 and/or Zaprinast, in particular wherein UK5099 is 2-cyano-3-(1-phenyl-1H-indol-3-yl)-2-propenoic acid. Most preferably, the MPC inhibitor is or comprises UK 5099.

In one embodiment, the immunotherapy comprises administering the MPC inhibitor to a subject. Without being bound by theory, the MPC inhibitor may increase the frequency of T-cells within a subject when administered to said subject, thus boosting and/or prolonging an immune response and/or extending the protective effect of vaccination. In some embodiments, the MPC inhibitor is administered to a subject for treating a different disease than cancer. As described above, the invention relates to an in vitro cell culture method comprising a step of contacting T-cells with an MPC inhibitor. In particular, the MPC inhibitor is comprised in the medium used for culturing the T-cells. Evidently, when the MPC inhibitor is added to an in vitro T-cell culture, i.e. to the culture medium used for culturing the T-cells, said T-cells are contacted with the MPC inhibitor. Thus, the T-cells are cultured in the presence of an MPC inhibitor according to the inventive method provided herein. In some embodiments, the MPC inhibitor is added to the culture medium before the T-cells are cultured with said medium. The MPC inhibitor may be also added to the culture after the T-cell culture is initiated, e.g. after or during the cells are seeded or incubated, but ideally shortly after culture start. It is preferred that the MPC inhibitor is present in the culture medium from the beginning of the culture. Moreover, preferably, the T-cells are at least contacted with the MPC inhibitor while being activated, although it is not strictly necessary that the MPC inhibitor is present during the entire activation phase.

Hence, the MPC inhibitor is used for culturing T-cells, as described herein, in particular for generating and/or maintaining T-cells with a memory phenotype.

Thus, the invention also relates to a method for generating and/or maintaining T-cells with a memory phenotype comprising the steps of culturing T-cells in vitro and adding an MPC inhibitor to the culture.

Accordingly, the invention further relates to an in vitro cell culture comprising T-cells and an MPC inhibitor. Said in vitro cell culture may comprise a culture vessel (e.g. a dish, a well plate, a bioreactor, a flask, or a bottle), a culture medium comprising an MPC inhibitor as provided herein, and T-cells. The culture may be static or dynamic (e.g. agitated by means of rotation or stirring).

In a preferred embodiment, the method of the invention further comprises a step of obtaining T-cells with a memory phenotype from the culture. Thus, the inventive methods provided herein may comprise a further step of obtaining the cells, in particular the T-cells, from the culture, thereby producing a cell population comprising T-cells with a memory phenotype, as described herein.

Preferably herein, the T-cells are human cells, for example, human umbilical cord blood (CB) cells and/or peripheral blood mononuclear cells (PBMC). Thus, the invention further relates to a cell population comprising human T-cells with a memory phenotype obtained by the method of the invention.

In a preferred embodiment, the T-cells are activated before and/or during culture, preferably while being cultured.

The term “memory phenotype”, as used herein, is defined as a cell state which resembles a memory T-cell at least in some aspects. The term “memory-like T-cell” is used herein interchangeably with the term “memory phenotype”. An important feature associated with a memory phenotype is the longevity of the cell. Longevity means that the cell or a progenitor survives long enough, e.g. without dividing or slowly dividing, in a subject to be able to elicit a therapeutic effect. In particular, a cell with a memory phenotype has stem cell-like properties. The longevity is preferably due to self-renewal which comprises proliferation. Self-renewal, as used herein, is not meant in a strict sense, but also includes the capacity to maintain a largely similar, although not necessarily identical, phenotype for a therapeutically relevant period of time. The self-renewal can be maintained for the entire life-time or even beyond, but it is sufficient, as used herein, if it is maintained long enough for the therapeutic purpose. A therapeutically relevant period of time means that the transferred cells or their progeny persist long enough in a subject to have a therapeutic effect.

While a T-cell with a memory phenotype is living, and preferably proliferating, it typically maintains the capacity to differentiate into effector cells. The capacity to generate an effector T-cell is thus another important feature associated with a memory phenotype. A T-cell with a memory phenotype thus can produce a higher number of therapeutically active effector cells than effector cells themselves which rather tend to senesce and die early. Important functional features associated with a memory phenotype can be also measured relative to other cell populations. For example, longevity, self-renewal and/or the capacity to differentiate into effector cells may be compared to effector cells, terminally differentiated cells and/or senescent cells.

Another important feature associated with the memory phenotype is the ability of the cells to react with an increased amplitude of (re)activation to a reencounter of the antigen, as is observed, e.g., with memory T-cells.

Memory T-cells are poised to respond to a reencounter of the antigen with a kinetics that is much faster than the primary response from a naive T-cell. Furthermore, memory cells are arrested in G1 of the cell cycle while naive cells are at G0. This allows for a rapid cell division, as they simply proceed through the cell cycle once recalled by a second encounter with antigen. Likewise, their effector genes are poised for rapid transcriptional activation which leads to a swift cytokine response and generation of a fresh cohort of effector T-cells. In addition, a small proportion of memory cells renew and continue to cycle slowly as a way to preserve the pool of memory cells. It is thought, based on serial adoptive transfer experiments, spanning a few years of experimentation, that this cycle can be repeated more than 30 times without sizable diminution of the memory cell state.

The “duality” of memory cells can be further described as follows: upon reactivation, memory cells mount a secondary rapid response and their progeny follow two fates: rapid generation of a new cohort of secondary (or tertiary if the second recall, or quaternary if a third recall) effectors (it is thought that the majority of reactivated memory cells follow this path) and self-renewal of memory cells which go on to preserve the memory pool (it is thought that this refers to a small proportion of the reactivated memory cells).

The amplitude of (re)activation may be characterized by an increased proliferation and/or production of pro-inflammatory and/or cytotoxic molecules/cytokines. For example, memory T-cells are capable of effectively producing the effector cytokines IFNγ and Tumor Necrosis Factor-α (TNF) and the proliferation-inducing cytokine IL-2 upon reencountering the specific antigen. This ability may be tested experimentally by restimulating the cells with Phorbol 12-Myristate 13-Acetate (PMA, a PKC activator) and Ionomycin (a Ca²+ ionophore, activating NF-κB and NFAT), as described previously (Tanchot (1998) 8(5):581-90).

It is also possible to restimulate memory T-cells by loading antigen-presenting cells with the cognate protein/peptide and co-culture those with the T-cells. However, such a restimulation assay depends on the previous (first) activation of the T-cells by the same cognate protein/peptide.

In contrast, restimulation with PMA and Ionomycin also works well for polyclonal T-cells, e.g. a pool of T-cells containing thousands of different TCRs recognizing at least as many different antigens. In other words, PMA and ionomycin allows for a non-TCR-specific global reactivation of the T-cells. Specifically, PMA and Ionomycin allow to (re)activate T-cells by providing the two signals that go downstream of the TCR signaling pathway, yet bypassing TCR engagement, i.e. PMA activates protein kinase C and Ionomycin is a calcium ionophore. PMA and Ionomycin together provide maximal activation of T-cells.

Thus, the restimulation with PMA and Ionomycin, allow to measure the effector functions of memory T-cells and/or T-cells with a memory phenotype, and the production of cytokines upon restimulation can thus be correlated with the memory differentiation state.

As described herein and demonstrated in the appended examples, the T-cells obtained by the method according to the invention, may be reactivated and respond with an increased magnitude/amplitude, i.e. when they have been generated and activated in the presence of an MPC inhibitor.

Thus, the cell population of the invention, i.e. the T-cells with a memory phenotype comprised therein, may have an unaltered or enhanced reactivation capacity. In particular, the reactivation capacity may be enhanced compared to the initial T-cells of the culture, e.g. naïve T-cells, and/or unaltered or enhanced compared to T-cells that have been generated the same way but without an MPC inhibitor and/or in the presence of an AKT inhibitor, as described herein.

A memory phenotype, as used herein, can also and/or additionally be defined by markers. Markers allow the distinction of a phenotype/cell state from another phenotype/cell state, for example a memory phenotype from an effector phenotype. A marker can be, for example, an RNA and/or protein whose presence or absence is associated with one or more important functional features. Such a marker may refer to the presence or absence of gene and/or protein expression and/or subcellular localization. This type of marker is typically described by “marker expression” or as “expression marker”. A marker can be also a functional property of a cell or cell population which can be determined by a standard assay. This type of marker is described as “functional marker”. Typical important functional features of a memory phenotype are mentioned herein, i.e. in the previous paragraph(s).

The presence or absence of a marker can be determined for a single cell and/or for a population of cells. Especially in case of a population of cells, suitable measurables are the average and median values and the frequency of cells with positive/negative and/or high/low values. For example, thresholds may be set and cells below a threshold may be considered “negative” for this marker (even if a numerically positive value is measured) and only cells above the threshold may be considered “positive”. More thresholds may be chosen to categorize cells, e.g., into low, mid and high marker expressing cells. This is further detailed out herein below. In some cases, a marker may also refer to another moment such as the variance.

Expression of a memory marker is associated with an important functional memory phenotype, whereas expression of a non-memory marker is associated with a cell state different from a memory phenotype. Absence or low expression of a memory marker is associated with a cell state different from a memory phenotype and absence or low expression of a non-memory marker may be associated with an important functional memory phenotype.

Presence and/or high expression of a marker can be described, for example, by the symbols “+”, “⁺”, “high”, “positive”, “pos”. Intermediate marker expression can be described, for example, by the symbols “+/−”, “^{+/-}”, “mid”. Absence and/or low expression of a marker can be described, for example, by the symbols “−”, “⁻”, “low”, “negative”, “neg”. Typically, the terms “presence of”, “positive” and “high” marker expression are used interchangeably. Typically, the terms “absence of”, “negative” and “low” marker expression are used interchangeably. In cases, where exact distinction between positive and high, or negative and low expression is explicitly required, only the terms “high” or “low” refer to high or low expression, respectively.

Marker expression can be determined by methods well-known in the art, for example, but not limited to, flow cytometry, mass cytometry (also commonly known as CyTOF), western blot, quantitative RT-PCR, in-situ hybridization, microarray, RNA sequencing, nanostring, mass spectrometry, and fluorescent fusion-proteins expressed from an endogenous gene locus. Other synonymous terms for quantitative RT-PCR, as uses herein, are “Q-PCR”, “quantitative PCR”, “quantitative real time RT-PCR” and “RT Q-PCR”. The terms “flow cytometry” and “FACS” are used interchangeably herein in the context of marker analysis. A preferred method is flow cytometry/FACS.

To determine if the marker is expressed, the sample is preferably compared to a negative control. Preferably, the marker is not detected in the negative control. The threshold level for positive marker expression is preferably based on data from prior art and/or the present invention showing a link between said expression level and an important functional feature. Positive or negative marker expression cannot be solely based on the presence or absence of one or very few RNA or protein molecule(s). The threshold for positive marker expression, as used herein, does not depend on the detection method. Similarly, negative or low marker expression should be based on the expression level which is associated with a functional feature. A negative marker expression, as used herein, cannot be classified as positive only because a more sensitive detection method is used. For example, the expression of a marker at the cell surface can be determined by staining with a specific antibody and flow cytometry. When the staining intensity is similar to an unspecific staining, for example IgG isotype control staining, or autofluorescence (unstained) control, then it can be classified as negative or low. If a cell which has such a low level of marker expression has certain functional properties, then this may be a useful “negative” marker even when a more sensitive method would allow detecting some marker molecules in the cell. However, a sensitive detection method may allow determination of further useful markers.

Functional markers can be determined by a standard assay. Preferably, a standard assay is commercially available and comprises a detailed protocol. It may further, but not necessarily, comprise an internal reference. A standard assay is typically easier to perform than an assay for determining a complex inherent feature such as longevity in vivo. Preferably, a standard assay may be accomplished by routine in vitro experimentation.

Marker expression can occur and/or be measured at the cell surface or within a cell (intracellular). Marker expression can be measured in living cells and/or in dead (fixed) cells. Cell surface markers can typically be measured in living cells, for example by flow cytometry/FACS. Living FACS-sorted cells may be further used, for example, for in vitro culture and/or transferred into a subject.

Many memory markers are known in the art. Some memory markers may be similar between species and/or cell types. Other memory markers may be differentially expressed between species and/or cell types. Relevant species are, for example, mouse, human and non-human primates. Relevant cell types are, for example, CD8+ T-cells, CD4+ T-cells and B-cells.

The memory phenotype, as used herein, is based on definitions used in the art. However, as the skilled person may know, those definitions may vary over time and between different laboratories/research groups/manufacturers. The more widely accepted a marker is and the more clearly and/or tightly it is linked to important functional properties of memory T-cells and/or B-cells, the more suitable this marker is.

The memory phenotype of T-cells, e.g. CD8+ T-cells or CD4 T-cells, as used herein, refers primarily to stem cell memory T-cells (T_{SCM}) and/or central memory T-cells (T_{CM}). It does not refer to effector memory T-cells (T_{EM}), effector T-cells (T_{EFF}) and/or terminally differentiated T-cells (T_{EMRA}). The preferred T-cell memory phenotype, e.g. CD8+ T-cell memory phenotype, refers to central memory T-cells (T_{CM}).

Suitable positive expression markers for the memory phenotype (memory markers), in particular of CD8+ T-cells, but also, at least partly, of CD4+ T-cells and/or B-cells, are, for example, CCR7, CD62L, CD27, CD28, CD127 and/or TCF1.

Preferred memory (expression) markers are CCR7, CD62L and TCF1, in particular CD62L and TCF1, or CD62L and CCR7.

A very preferred memory (expression) marker is CD62L. CD62L is particularly useful for distinguishing T-cells with a memory phenotype from naïve T-cells and effector T-cells, preferably complemented by CD44 or CD95 expression.

It has been further found in the context of the invention that an open chromatin configuration, e.g. in or in the vicinity of genes associated with the expression of the memory T-cell differentiation program, may be used as a marker of the memory phenotype as used herein, i.e. for T-cells. In particular, the open chromatin configuration may be characterized by an increased trimethylation on the lysine 4 residue of histone 3 (H3K4-3Me), an increased acetylation on lysine 27 residue of histone 3 (H3K27-Ac) and/or more accessible chromatin regions. The accessible chromatin regions may be determined by methods known in the art such as ATAC-seq, DNAse-Seq or MNase-Seq, e.g. as demonstrated in the appended Examples. H3K4-3Me and/or H3K27-Ac levels and their associated genes may be also determined by methods known in the art, e.g. by western blotting, immunostaining, ChIP and/or ChIP-seq.

To further distinguish T-cells and/or B-cells with a memory phenotype from naïve T-cells, the auxiliary markers CD122, CD95 and/or production of IL-2 and/or IFN-gamma may be used. Those auxiliary markers, however, are not sufficient by themselves.

A suitable negative expression marker for the memory phenotype (non-memory marker), in particular of CD8+ T-cells, but also, at least partly, of CD4+ T-cells and/or B-cells, is KLRG1.

A further suitable marker set for human central memory T-cells (comprised in “memory phenotype”) is: CCR7⁺/CD27⁺/CD28⁺/CD45RA^{neg}. Alternatively, the CD45RA expression may be low instead of negative. Furthermore, the human central memory T-cells may express CD45RO.

A suitable marker set for human central effector memory T cells (not comprised in “memory phenotype”) is: CCR7^{neg}/CD27^{+/-}/CD28^{+/-}/CD45RA^{neg}.

Suitable markers of T-cell differentiation, in particular memory markers and non-memory markers are also disclosed in Gattinoni et al., Nat Rev Cancer. 2012 October; 12(10):671-84 and Kishton et al., Cell Metab. 2017 Jul. 5; 26(1):94-109.

Effector memory T-cells, effector T-cells and/or terminally differentiated T-cells, all which are not comprised in the term “memory phenotype” can be characterized by negative or low expression of CCR7, CD62L, CD27 and/or CD28, preferably in combination with positive or high expression of CD122, CD95 and/or KLRG1.

Effector T-cells are typically specialized for one or more specific functions, for example cytotoxicity, secretion of cytokines and/or activating or repressive modulation of other immune cells.

Naïve T-cells can be characterized by high or positive expression of CCR7, CD62L, CD27, CD45RA and/or CD28 and absence of CD122, CD95, IL-2R-beta and/or KLRG1. Furthermore, naïve T-cells are not cytotoxic and typically do not secret IL-2 and/or IFN-gamma. Naïve T-cells and/or B-cells have the capacity to differentiate into memory cells and/or effector cells. They have typically not been stimulated by antigen and a second stimulus. Naïve T-cells typically continuously recirculate between blood and the secondary lymphoid organs. It is in these organs that they may be presented with their cognate antigen by an antigen presenting cell following which they are activated. This event is also commonly referred to as “priming”. Following this event, they typically proliferate, acquire functional competencies, e.g. effector functions, and migrate to non-lymphoid tissues.

Thereby, naïve T-cells develop either into terminally differentiated short-lived effector cells (SLECs), or into memory precursor effector cells (MPECs) which can further differentiate into both, central memory T-cells (expressing CD62L) and effector memory T-cells (lacking expression of CD62L).

Central memory T-cells, descending from the memory precursor effector cells and staying throughout the memory phase, typically reside, like naïve cells, in secondary lymphoid organs. Upon re-exposure to antigen, these cells undergo activation, enhanced proliferation and rapid expression of effector functions. This event is commonly referred to as recall response or secondary response. While the majority of memory T-cells rapidly express effector functions, a variable but small fraction of them remains in the memory state of differentiation. This characteristic is called self-renewal. These are the dynamics of memory T-cells that are reminiscent of, and similar to, stem cells.

T-cells upregulate the central memory marker CD62L when cultured according to the invention. However, when the T-cells are transferred into a subject where they reencounter the antigen, they get reactivated. Upon (re-)activation in vivo, the T-cells quickly lose the CD62L marker, which however gets re-expressed when the T-cells differentiate into central memory T-cells again in vivo. Thus, the generation of memory precursor effector T-cells (MPECs) and/or central memory T-cells in vivo, for example in the spleen, upon transfer of the in vitro cultured T-cells into a subject, is a further characteristic of the memory phenotype.

Further suitable functional memory markers may be based on metabolic changes which are known in the art to occur during the activation and/or differentiation of T-cells and/or B-cells. It is well known in the art that, for example, T-cells change from a mitochondrial metabolism and fatty acid oxidation towards an anabolic metabolism with aerobic glycolysis, see for example Kishton et al., Cell Metab. 2017 Jul. 5; 26(1):94-109. T-cells and/or B-cells with a memory phenotype can be distinguished from the respective effector cells by differences in oxygen consumption rates. In particular T-cells with a memory phenotype can be distinguished that way from effector memory T-cells (T_{EM}), effector T-cells (T_{EFF}) and/or terminally differentiated T-cells (T_{EMRA}).

T-cells and/or B-cells with a memory phenotype typically have an increased basal oxygen consumption, maximal respiratory capacity and/or spare respiratory capacity compared to the respective effector cells.

The basal oxygen consumption, maximal respiratory capacity and/or spare respiratory capacity can be measured, for example, with a XF-96 Extracellular Flux Analyzer (Seahorse Bioscience) in combination with suitable protocols and drugs. Suitable protocols are known in the art and can be obtained, for example, from Seahorse Bioscience. Addition of oligomycin allows to calculate the ATP-linked respiration by subtracting the oligomycin OCR from basal OCR. FCCP enables the electron transport chain (ETC) to reach its maximal rate, allowing to determine the cellular maximal respiratory capacity. Finally, rotenone and antimycin A inhibit the electron transport chain which indicates the non-mitochondrial respiration. The spare respiratory capacity (SRC) is defined as the difference between basal OCR and maximal OCR obtained after FCCP addition. The terms “basal oxygen consumption rate”, “basal oxygen consumption”, “basal respiration” and “basal OCR” are used interchangeably herein. The terms “maximal respiratory capacity”, “maximal respiration” and “MCR” are used interchangeably herein. The terms “spare respiratory capacity” and “SCR” are used interchangeably herein.

Activation of T-cells and/or B-cells typically occurs when an antigen is recognized by a TCR or BCR at the cell surface and usually only when a second stimulus is present. The second stimulus may be provided by APCs or other immune cells, in particular CD4+ helper T-cells and typically by additional protein-protein interactions at the cell surfaces. Typical second stimuli for T-cells provided by APCs are for example, 4-1BBL, CD80, CD86 and/or OX40L. Second stimuli for CD8+ T-cells may be provided by CD4+ T-cells, in particular helper T-cells. CD4+ T-cells and B-cells may provide each other with second stimuli.

Activation of T-cells and/or B-cells generally leads to the initiation of coupled processes of proliferation, differentiation and migration. Thus those activated cells differentiate and/or mature to perform specific tasks for eliminating an antigen and/or its source. Activation is typically followed by proliferation of the cells and/or movement to a different tissue. During differentiation and/or maturation T-cells change their phenotype. They also change their function. CD8+ T-cells may become cytotoxic, and CD4+ T-cells may differentiate into different subsets which may either support or repress the immune response. Activation usually involves secretion of cytokines to modulate the function of other immune cells.

Activation of T-cells in vitro, as used herein, can be accomplished by methods known in the art, and may also refer to the “priming” of T-cells. For example, T-cells may be activated by APCs, and/or by a mix of anti-CD3 and anti-CD28 antibodies. The anti-CD3 and anti-CD28 antibodies may be in solution, coupled to beads and/or attached to the surface of antigen presenting cells. In particular, the APCs may be used together with an antigenic peptide that is presented by the APCs.

Antigen-presenting cells that are particularly useful for the activation of T-cells may be dendritic cells, macrophages, B-cells, preferably dendritic cells, and/or artificial antigen presenting cells described in the art for this purpose; see e.g. Neal et al. (2017) J. Immunol. Res. Ther. 2(1):68-79.

Furthermore, the TCR and costimulatory receptor may be stimulated with anti-CD3/CD28 antibody beads, as illustrated in the appended Examples. Provision of a specific antigen, for example in case of OT1 T-cells, the ovalbumin N4 peptide (SIINFEKL) and/or provision of IL-2 may boost the activation/differentiation/maturation of T-cells. In particular, the activation of T-cells, i.e. naïve T-cells, starts when the T-cells are contacted with an antigenic peptide (i.e. in combination with APCs), and/or a mix of anti-CD3 and anti-CD28 antibodies, and preferably IL-2. IL-2 may be present in solution or attached to the surface of antigen presenting cells. Evidently, the anti-CD3 and anti-CD28 antibodies must be suitable for stimulating the TCR and costimulatory receptor responsible for T-cell activation. Furthermore, other molecules binding to said receptors may be also suitable in the context of the invention, if they stimulate T-cell activation.

In the in vitro culture method of the present invention, T-cell and/or B-cell activation and differentiation into memory T-cells occur preferably side-by-side. Thus, the MPC inhibitor is preferably present in the culture medium together with the activating agent(s), i.e. an antigenic peptide (i.e. in combination with APCs), and/or a mix of anti-CD3 and anti-CD28 antibodies, and preferably IL-2.

The terms “differentiation” and “maturation”, as used herein, are not sharply separated. Differentiation, however, rather refers to cell states which maintain some plasticity, i.e. they may still give rise to different cell types, whereas the cell fate of cells which are maturing is more determined.

Suitable activation markers, as used herein, are CD25, CD44, CD71 and/or CD98. An additional activation marker is the extracellular acidification rate (ECAR) which can be measured by methods known in the art, for example with XF-96 Extracellular Flux Analyzer (Seahorse Bioscience) and/or in parallel with the oxygen consumption rate.

Suitable markers for the reactivation capacity of T-cells with a memory phenotype, i.e. upon reencounter of the antigen or a restimulation assay mimicking such a reencounter, as used herein, are IFNγ, TNF and/or IL2, preferably IFNγ. Since these markers are molecules that are normally secreted into the culture medium, a golgi inhibitor (e.g. Brefeldin A, BFA) can be applied during the restimulation assay. Application of a golgi inhibitor leads to the intracellular accumulation of these cytokines and thus allows for their measurement by flow cytometry or imaging. A normal, high or enhanced reactivation capacity, i.e. efficient expression of IFNγ, TNF and/or IL2, preferably IFNγ, may be used as a memory marker, i.e. a functional memory marker, as described herein.

Positive memory expression markers, negative memory expression markers, functional memory markers, and activation markers can be combined to describe a cell population obtained by a method of the invention. Furthermore, the cell population may be characterized by the chromatin configuration, i.e. whether it is in an open configuration, as described herein.

The method of the invention allows maintaining a memory phenotype of T-cells and/or B-cells subjected to in vitro culture and/or allows generating T-cells and/or B-cells with a memory phenotype during in vitro culture. The term “generating” refers to differentiation from more naïve T-cells and/or B-cells. Expansion of the cells during in vitro culture is preferred, but not required.

Cells as used herein, when not specified explicitly or by context, refer to T-cells. In a preferred embodiment of the invention, the cells are T-cells. In a very preferred embodiment, the T-cells comprise cytotoxic T-cells.

T-cells are lymphocytes of the adaptive immune system. They are derived from hematopoietic stem cells. T-cells are important for cell-mediated immunity and B-cells are important for humoral immunity. Both are subjected to careful selection and tuning of their reactivity during ontogeny. The result is an immune system populated by naïve T-cells and B-cells which are largely depleted of autoreactive cells. There are two major subclasses of T-cells: CD4+ T-cells and CD8+ T-cells. CD8+ T-cells may develop into cytotoxic T-cells. Their major function is to destroy virus-infected cells and tumor cells. The terms “CD8+ T-cells” and “cytotoxic T-cells” are used herein interchangeably. Cytotoxic T-cells, as used herein, refer to CD8+ T-cells. As used herein, the term “cytotoxic T-cells” does not necessarily refer to an advanced differentiation stage but includes naïve CD8+ T-cells and/or memory CD8+ T-cells. CD4+ T-cells may, in some cases, also acquire cytolytic activity.

There are various subsets of CD4+ T-cells. A major group of CD4+ T-cells are helper T-cells. Helper T-cells are often further grouped into different subsets of cells with specialized functions:
Th1: Produce an inflammatory response, key for defense against intracellular bacteria, viruses and cancer.
Th2: Aid the differentiation and antibody production by B cells.
Th17: Defense against gut pathogens and at mucosal barriers.
Th9: Defense against helminths.
Tfh: Help B cells produce antibody.

Another major group of CD4+ T-cells are regulatory T-cells. Regulatory T-cells are crucial for the maintenance of immunological tolerance. Their major role is to shut down T-cell-mediated immunity toward the end of an immune reaction and to suppress autoreactive T-cells that escaped the process of negative selection in the thymus.

Both, naïve CD8+ T-cells and CD4+ T-cells can differentiate into memory T-cells. There are different sets of memory T-cells which have in common that they are long-lived and can quickly expand to large numbers of effector T cells upon re-exposure to their cognate antigen. As used herein, memory T-cells or T-cells with a memory phenotype do not comprise effector memory T-cells, but rather refer to stem cell memory T-cells and/or central memory T-cells.

A major function of B-cells is the generation of antibodies. Other functions include the presentation of antigens and production of cytokines. Memory B-cells are often dormant. Their function is to circulate through the body and initiate a stronger, more rapid antibody response (known as the anamnestic secondary antibody response) if they detect the antigen that had activated their parent B cell. Effector B-cells include plasmablasts and plasma cells.

In certain embodiments, the cells may be a mix of CD8+ T-cells, CD4+ T-cells and/or B-cells. They may be mixed before, during or after in vitro culture and/or within a subject (before combined adoptive cell transfer and/or upon separate adoptive cell transfers).

In certain embodiments, the cells are mammalian cells. In a preferred embodiment, the cells are human, non-human primate or mouse cells. In a very preferred embodiment the cells are human cells. In one embodiment, the cells are cells from pets such as cat cells or dog cells.

The terms “subject”, “patient” and “living organism” are used interchangeably herein and refer to a mammal, preferably a human, non-human primate or mouse, very preferably a human, which is the source of cells and/or the recipient of cells of the invention. Preferably, the subject is both source and recipient of the cells.

In certain embodiments, the memory phenotype comprises expression of one or more memory marker(s).

In certain embodiments, the memory phenotype comprises absence of expression of one or more non-memory marker(s).

Thus, the memory phenotype according to the invention, i.e. in the context of T-cells, preferably CD8+ T-cells, may comprise expression of at least one memory marker selected from the group consisting of: CD62L, TCF1, CD27, CD127, CCR7 and CD28. A further memory marker may be the nuclear localization of FOXO1. Furthermore, the memory phenotype may comprise absence of detectable expression of the non-memory marker KLRG1. Preferably, the memory phenotype comprises expression of the memory marker(s) CD62L and/or TCF1, preferably CD62L. Very preferably, the memory phenotype comprises surface expression of the memory marker CD62L.

In one embodiment, i.e. in the context of human T-cells, the memory phenotype comprises expression of CCR7, CD27, CD28 and no or low, preferably low, expression of CD45RA.

In certain embodiments, expression of memory marker(s) and/or expression of non-memory markers is compared to a DMSO control. In particular, this means that a memory marker is upregulated compared to the DMSO solvent control, and a non-memory marker is downregulated compared to the DMSO solvent control.

Furthermore, the T-cells according to the invention may express at least one activation marker, preferably wherein said at least one activation marker is selected from the group consisting of: CD25, CD44, CD71 and CD98.

Thus, in a particular embodiment, T-cells generated/maintained/obtained by a method of the present invention show surface expression of CD25, CD44, CD71, CD98 and increased surface expression of CD62L.

In certain embodiments, the memory phenotype comprises an open chromatin configuration as described herein. In particular, the open chromatin configuration may be characterized by an increased trimethylation on the lysine 4 residue of histone 3 (H3K4-3Me), an increased acetylation on lysine 27 residue of histone 3 (H3K27-Ac) and/or more accessible chromatin regions compared to the respective parameters in a control cell population, wherein the control T-cells have not been contacted with an MPC inhibitor.

In a preferred embodiment, the T-cells and/or B-cells are autologous cells. Autologous cells, as used herein, refer to cells which are derived from a subject which is to be treated with those cells, preferably after in vitro culture and/or manipulation.

In certain embodiments, the T-cells and/or B-cells comprise cells derived from tumor-infiltrating lymphocytes (TILs). Preferably, the TILs are T-cells. TILs may reside within a tumor of the patient which is to be treated with TILs with a memory phenotype obtained by the invention. Methods are known in the art to isolate TILs from the tumor of a patient. Isolated TILs are cultured by the method of the invention, in particular for adoptive cell transfer into said patient.

In one embodiment, the T-cells comprise T-cells that are derived from tumor-draining lymph node cells.

The term “derived”, as used herein, means that the cells have altered their cell state and/or environment/location, in particular that they are isolated from a subject and grown in vitro or ex vivo. Preferably, the derived T-cells maintain at least one characteristic from the T-cells from which they are derived, e.g. characteristic parts of the genome (such as the specific TCR gene), and/or the antigen specificity.

In certain embodiments, the T-cells are derived from splenocytes and/or circulating blood cells. Further suitable sources of T-cells and/or B-cells are, for example, bone marrow, lymphoid organs, lymph, thymus and/or tissues infected by a parasite, bacteria and/or virus. For example, the T-cells used for culturing according to the invention may be cells that have been obtained from the spleen, blood, e.g. cord blood, a lymph node, i.e a tumor-draining lymph node, or from a tumor. Further suitable sources of T-cells are induced pluripotent stem cells (iPSCs) as described in Temelli (2015), Cell Stem Cell 16(4):357-66 and/or in Nianias (2019), Curr Hematol Malig Rep 14(4):261-268.

The terms “in vitro” and “ex vivo” are used interchangeably herein and refer to the culture of cells in a culture dish, plate, flask and/or bottle outside of a living organism. Cell culture comprises a suitable liquid culture medium which allows the cells to survive and maintain and/or generate the desired phenotype and/or cell state. The cells may have been cultured and/or frozen previously. Preferably, the cells are freshly obtained from a subject before subjected to the in vitro culture of the invention.

A suitable cell culture medium for the culture of T-cells is, for example, based on RPMI medium (basal medium). Preferably, said medium further comprises fetal-calf or human serum, Penicillin/Streptomycin, 8-mercaptoethanol, HEPES, Non-essential amino acids, L-glutamine, Sodium Pyruvate, IL-2 and an antigenic peptide. Preferably, IL-7 is added during culture. Other media suitable for culturing T-cells in cell manufacturing facilities may be based on commercially available basal media that limit the amount of fetal-calf or human serum while privileging inclusion of albumin, e.g. AIM V Serum Free Medium (Thermo Fisher Scientific).

For example, a suitable basal medium for culturing human T-cells may be RPMI medium supplemented with 10% human serum, penicillin (e.g. 50 IU/ml), streptomycin (e.g. 50 μg/ml), L-glutamine (e.g. 4 mM), non-essential amino acids (e.g. 1% (v/v)) and 2-mercaptoethanol (e.g. 50 μM).

Evidently, the culture medium according to the invention may comprise an MPC inhibitor as described herein.

Furthermore, the culture medium may further comprise further compounds according to the invention, e.g. IL-2, IL-7, an antigenic peptide, and/or anti-CD3 and anti-CD28 antibodies as described herein.

The meaning of adding a compound, for example an MPC inhibitor, IL-2, an antigenic peptide and/or IL-7, to the culture, culture medium and/or during in vitro culture, as used herein, is identical to contacting the cells with said compound during culture. As described herein, the compound may be added to the culture or culture medium at the beginning of the culture or after the culture has been initiated, preferably at the beginning of the culture. The terms “culture”, “cell culture”, “in vitro culture”, “culture medium” all refer to the in vitro or ex vivo environment where cells can be contacted with said compound, in particular an MPC inhibitor.

In a preferred embodiment, the T-cells comprise naïve cells which acquire a memory phenotype.

In a preferred embodiment, the T-cells are expanded during culture. Expansion means that more T-cells exist at the end of the in vitro culture than at the start, e.g. as a result of cytokine-driven T-cell proliferation.

In certain embodiments, the T-cells comprise a heterologous antigen receptor. The term “heterologous”, as used herein, refers to a gene or gene variant/allele which does not naturally occur in a certain cell. An antigen receptor as used herein, is a protein, typically expressed on the cell surface, which recognizes a specific antigen. A heterologous antigen receptor, as used herein, is thus a protein which binds to a specific antigen, which is expressed in a (host) cell, and which is encoded by a gene that has been introduced into said cell. Preferably the host cell is a T-cell. Very preferably, the host cell is a T-cell.

Preferably, the gene is stably integrated into the genome. Methods to stably integrate a gene into the genome of a host cell and to express this gene in that host cell are well known in the art. The recombinant DNA construct comprising the gene, in particular the gene encoding for a heterologous antigen receptor, further comprises a promoter which allows expression in the host cell. The promoter can be constitutively active, which means that it allows gene expression in nearly all cell types of the organism. A constitutive promoter is for example, but not limited to CAG, SFFV, PGK or CMV. The promoter can be also specific to the tissue of the host cell and/or related cells, for example cells into which the host cell can differentiate. A tissue specific promoter is, for example, a naturally occurring or modified promoter of a gene which is strongly expressed in said tissue(s). The recombinant DNA construct can be delivered by methods known in the art. The host cell can be, for example, electroporated, transfected, nucleofected and/or transduced with the recombinant DNA construct. Suitable transfection methods are, for example, based on lipofection, such as lipofectamine, or cationic polymers such as Polyethylenimine (PEI). The recombinant DNA construct may further comprise sequences for transposon mediated integration into the genome. When introduced together with a plasmid encoding the corresponding transposase into a host cell, the gene comprised in the recombinant DNA construct can be integrated into the genome of the host cell. Known transposon systems are, for example, piggyBAC or sleeping beauty. Preferably, the host cell is transduced with a viral vector comprising the recombinant DNA construct. Suitable transduction methods comprise, for example, retroviruses, lentiviruses, adenoviruses and/or adeno-associated viruses. The recombinant DNA construct may further comprise sequences required for virus production and/or integration of the gene into the genome of the host cell. Methods to generate viruses comprising the recombinant DNA construct, or an RNA variant thereof, are known in the art. Preferably, the virus, for example a lentivirus, mediates integration of the gene into the genome of the host cell.

In a preferred embodiment, the heterologous antigen receptor is a T-cell receptor (TCR). A specific heterologous TCR may be identical to a naturally occurring TCR and may or may not be expressed also naturally in the host cell, preferably the host T-cell. Typically, the heterologous TCR has been identified and/or selected by methods known in the art. Identification and/or selection methods comprise predicting and/or measuring the binding of the TCR to an MHC/HLA-antigen complex and/or isolating cells expressing a TCR. The antigen may be specific for the patient, for example for the tumor of a patient. A tumor specific antigen is also called TSA or neoantigen. The presence of an antigen may be also associated with a tumor, but not necessarily be tumor-specific. The interaction of a TCR with an MHC/HLA-peptide complex is typically patient specific, because the patient expresses a certain set of MHC/HLA alleles and often also specific antigens. A heterologous TCR is particularly useful for adoptive transfer of T-cells expressing this TCR into a patient. Particularly effective may be further vaccination with an antigen, for example an antigenic peptide or an antigen-presenting cell presenting the antigen by MHC/HLA molecules. Preferably, the T-cells are autologous T-cells which express a heterologous TCR that has been selected to efficiently bind to a patient-specific MHC/HLA-peptide complex comprising a patient-specific antigen, for example a neoantigen and/or a tumor-associated antigen.

In a preferred embodiment, the heterologous antigen receptor is a chimeric antigen-receptor (CAR). As used herein, a CAR refers to an artificial T-cell receptor. Chimeric antigen receptors combine many facets of normal T cell activation into a single protein. They link an extracellular antigen recognition domain to intracellular signaling domains, which activates the T cell when an antigen is bound. CARs are composed of three regions: the ectodomain, the transmembrane domain, and the endodomain.

The ectodomain is the region of the receptor that is exposed to the outside of the cell and so interacts with potential target molecules. It consists of 3 major components: an antigen recognition region that binds the target molecule, a signal peptide that directs the nascent protein into the endoplasmic reticulum, and a spacer that makes the receptor more available for binding. The antigen recognition region consists, for example, of a single-chain variable fragment (scFv). An scFv is a chimeric protein made up of the light (VL) and heavy (VH) chains of immunoglobins, connected with a short linker peptide. The transmembrane domain is a structural component, consisting of a hydrophobic alpha helix that spans the cell membrane, for example a CD28 transmembrane domain. The endodomain is the internal cytoplasmic end of the receptor that perpetuates signaling inside the T cell. The endodomain is, for example, based on CD3-zeta's cytoplasmic domain. The endodomain typically also includes one or more chimeric domains from co-stimulatory proteins such as CD28, 4-1BB (CD137), or OX40.

An MPC inhibitor, as used herein, is a compound or a mix of compounds which inhibits the solute carrier, mitochondrial pyruvate carrier (MPC). Inhibition means that the function of the carrier is blocked or impaired.

In some embodiments, the MPC inhibitor allosterically impairs the carrier activity in particular by binding to the MPC protein.

In some embodiments, the MPC inhibitor may inhibit the production of the MPC protein, for example by inhibiting the transcription and/or translation of the MPC gene and/or mRNA.

In a preferred embodiment, the concentration of the MPC inhibitor is high enough to inhibit the transporter reaction of wild-type MPC.

In a preferred embodiment, the MPC inhibitor comprises one or more small molecule(s). A small molecule, as used herein, refers to a chemical compound, in particular an organic chemical compound, with low molecular weight, in particular less than 900 daltons.

Preferably, the small molecule MPC inhibitor inhibits the activity of MPC.

In a very preferred embodiment, the small molecule(s) comprise(s) UK5099, Pioglitazone, Rosiglitazone, MSDC-0602, MSDC-0160 and/or Zaprinast, in particular wherein UK5099 is 2-cyano-3-(1-phenyl-1H-indol-3-yl)-2-propenoic acid.

In some embodiments, the MPC inhibitor blocks the enzyme activity of MPC by 20, 40, 60, 80 or 100%. Preferably, the enzyme activity is blocked by at least 60%. Very preferably, the enzyme activity is blocked by at least 80%.

In some embodiments, the MPC inhibitor comprises an oligonucleotide or a precursor thereof, which interferes with MPC RNA. For example, the MPC inhibitor may comprise as oligonucleotide a small-interfering RNA (siRNA), a short-hairpin RNA (shRNA), an antisense RNA and/or the oligonucleotide functions by RNA-interference (RNAi), in particular to inhibit the production of MPC in a cell, for example by degrading the MPC mRNA and/or inhibiting the translation of MPC mRNA. A CRISPR/Cas9 editing technology may also be used, targeting the MPC-1 gene.

In some embodiments, the MPC inhibitor comprises an antibody and/or monobody. An (anti-MPC) antibody and/or monobody, as used herein, inhibits the activity of MPC by binding to the MPC protein. Preferably, the anti-MPC antibody and/or monobody is a therapeutic antibody. Therapeutic antibodies are well known in the art, and may, for example, be humanized antibodies and/or have improved pharmacokinetic properties such as improved half-life if the blood plasma and or lead to enhanced clearance of MPC. A monobody, as used herein, refers to a synthetic binding protein that is constructed using a fibronectin type III domain (FN3) as a molecular scaffold.

In some embodiments, the T-cells are contacted with the MPC inhibitor from the beginning of the culture and/or activation. In some embodiments, the T-cells are contacted with the MPC inhibitor during the entire culture period.

Preferably herein, the T-cells are contacted with the MPC inhibitor at least during activation, albeit it is not strictly required that the MPC inhibitor is present during the entire activation (priming) phase.

In some embodiments, the method of the invention further comprises a step of adding IL-2 to the culture. IL-2, as used herein, refers to interleukin-2.

In some embodiments, the method of the invention further comprises a step of adding one or more antigenic peptide(s) to the culture. Thus, the T-cells may be activated by contacting them with an antigenic peptide, in particular in the presence of antigen-presenting cells. An antigenic peptide, as used herein, is recognized by T-cells via a TCR.

An antigenic peptide is, in particular, a short linear peptide, e.g. 8 to 18 amino acids in length, that can be presented by a MHC.

Typically, an antigenic peptide that is presented by a class I MHC molecule is 8 to 10 amino acids in length. Antigenic peptides that are presented by a class I MHC molecule may be particularly useful for generating and/or maintaining CD8+ T-cells with a memory phenotype according to the invention.

Typically, an antigenic peptide that is presented by a class II MHC molecule is 13 to 25 amino acids in length. Antigenic peptides that are presented by a class II MHC molecule may be particularly useful for generating and/or maintaining CD4+ T-cells with a memory phenotype according to the invention.

In one embodiment, the antigenic peptide(s) is/are specific for the activation of a specific T-cell, T-cell clone and/or group of T-cells.

Furthermore, the T-cells may be activated by contacting them with anti-CD3 and anti-CD28 antibodies.

B-cells may be also activated by antigens other than antigenic peptides, including proteins, haptens and any other molecule to which antibodies can be made to such as, inter alia, carbohydrates, lipids, or glycolipids.

IL-2 and/or an antigenic peptide may enhance the activation of T-cells and/or their differentiation into cells with a memory phenotype. For example, adding IL-2 to the culture medium may promote cell expansion, production of IFN-gamma and/or cytotoxicity of CD8+ T-cells. The antigenic peptide may further promote selection of T-cells and/or B-cells which specifically recognize an antigen comprised in said peptide. Thus, the T-cells may be further contacted with IL-2, preferably together with the MPC inhibitor, preferably further together with the antigenic peptide and/or the anti-CD3 and anti-CD28 antibodies.

Thus, in a preferred embodiment, the T-cells are cultured in a medium comprising the MPC inhibitor, IL-2, and anti-CD3 and anti-CD28 antibodies and/or an antigenic peptide.

In some embodiments, the method of the invention further comprises a step of adding IL-7 to the culture medium. IL-7, as used herein, refers to interleukin-7.

Addition of IL-7 to the culture medium may promote the differentiation T-cells into cells with a memory phenotype and/or the survival of the cells, in particular of cells with a memory phenotype (Raeber (2018) Immunol Rev 283(1):176-193). In particular, IL-7 may favor the survival of the T-cells in vivo upon adoptive transfer but IL-7 is not absolutely necessary for obtaining the T-cells with a memory phenotype upon MPC inhibition. Without being bound by theory, IL-7 induces glycerol channel AQP9 expression in CD8+ T cells which enhances triglyceride synthesis to promote memory CD8+ T cell survival; Cui et al., Cell. 2015 May 7; 161(4):750-61.

In one embodiment, IL-7 is added to the culture medium after the MPC inhibitor and the antigenic peptide are washed out.

In a particular embodiment, the method further comprises the steps of
a. adding IL-2, an MPC inhibitor and an antigenic peptide to the culture medium for several days,
b. washing out the MPC inhibitor and the antigenic peptide and
c. adding IL-2 and IL-7 to the culture medium for several more days.

The time depends on the animal species and cells used. For example, when mouse CD8+ T-cells are used, step a of this embodiment takes about three days and step c about four days. The skilled person is able to select suitable time periods for cells from other animal species, in particular for non-human primates or humans where biological processes such as cell differentiation take more time.

In other words, the T-cells may be cultured in a first medium comprising the MPC inhibitor, IL-2, and anti-CD3 and anti-CD28 antibodies and/or an antigenic peptide, and then in a second medium comprising IL-2 and IL-7. However, it is also possible that the cells are just cultured in said first medium, as described herein.

As already described above, the invention further relates to a cell population comprising T-cells with a memory phenotype obtained by the inventive method provided herein. Preferably, the T-cells are human cells. Accordingly, the method of the invention may be further characterized by producing the inventive cell population provided herein. The cell population of the invention can be produced by the inventive method provided herein, as demonstrated in the appended Examples. However, it remains possible that the inventive cell population can be also produced in the future by other methods yet to be developed, e.g. by modifying the inventive method provided herein. Evidently, the cell population of the invention is or can be isolated. In particular, a cell population obtained by an in vitro culture method is inherently separated from other cells by the culture vessel and may be kept separate from other cells.

As indicated above, it has been, inter alia, surprisingly found in the context of the present invention that a human cell population highly enriched for T-cells with a memory phenotype could be obtained by culturing a cell population comprising naïve T-cells (e.g. human cord blood lymphocytes) in the presence of an MPC inhibitor. As known in the art and described herein, T-cells with a memory phenotype are therapeutically more effective, when administered to a patient, compared to other T-cells, e.g. effector T-cells. However, it is not possible to obtain T-cells with a memory phenotype directly from patients, i.e. not at therapeutically effective numbers, and further manipulate them (e.g. transduction with a TCR or CAR) and put them back into a patient, without using a suitable in vitro culture method that promotes and/or maintains the memory phenotype of the cells, and preferably allows expansion of the cells. It is thus evident that an in vitro culture method for generating and/or maintaining T-cells with a memory phenotype is required for effectively using T-cells with a memory phenotype for therapy, i.e. immunotherapy. In other words, a cell population isolated from a subject/human comprising memory T-cells with a memory phenotype is not suitable for therapy without culturing the cell population in vitro. Thus, in practice, any cell population comprising T-cells with a memory phenotype, i.e. at a high frequency such as at least 30%, 40%, 50%, 60%, 70%, 80%, or 90%, is obtained by a method comprising in vitro culture of the cells. In other words, the inventive cell population provided herein is only obtainable by a method comprising in vitro culture. Moreover, the inventive cell population provided herein is suitable for use in therapy, and thus may be used for therapy, i.e. immunotherapy.

The cell population of the invention is, in particular, a cell population, wherein at least 90%, 95%, 98% or 99% of the cells in the population are T-cells, and/or at least 30%, 40%, 50%, 60%, 70%, 80%, or 90%, or at least 95%, 97%, 98% or 99%, preferably at least 70%, of the cells in the population are T-cells with a memory phenotype. Preferably, said T-cells with a memory phenotype express CD62L.

Yet, the cell population of the invention may not be simply produced by enriching for T-cells with a memory phenotype obtained from another cell culture based on markers. Even if some markers, i.e. surface markers such as CD62L, could potentially allow to enrich for T-cells with a memory phenotype, such an approach may not allow to generate the cell population of the invention because the cells may be still functionally different (and/or different for the expression of other factors). Furthermore, merely enriching T-cells with a memory phenotype by sorting a culture comprising such cells based on memory marker expression (e.g. with FACS) may not provide to sufficiently large number of purified cells and may thus not be suitable for therapy, i.e. if T-cells with a memory phenotype only constitute a small proportion of the cells in the culture. Moreover, the sorting procedure usually leads to a cell loss for technical reasons which is counter the preferred aim to expand the T-cells with a memory phenotype, i.e. for therapeutic applications.

Thus, the cell population of the invention may be obtained by the inventive culture method provided herein without a cell sorting step such as FACS or MACS.

Thus, in one embodiment, the cell population of the invention is not purified based on marker expression, e.g. for CD62L positive T-cells, and/or not obtained by such a purification.

In one embodiment, at least 90%, 95%, 98% or 99% of the cells in the inventive cell population provided herein are CD8+ T-cells.

In one embodiment, at least 90%, 95%, 98% or 99% of the cells in the inventive cell population provided herein are CD8+ T-cells or CD4+ T-cells.

Despite the fact that T-cells, CD4+ T-cells or CD8+ T-cells can be readily purified by methods known in the art, e.g. by using suitable antibodies and flow cytometry, the inventive cell population provided herein can be obtained, and is preferably obtained, without such further purification.

However, in some embodiments, 90%, 95%, 98%, 99%, preferably 99.5% or 99.9% of the cells in the cell population are T-cells, CD8+ T-cells, or CD4+ T-cells, wherein such a cell population is obtained by further purifying the cell population obtained by the inventive in vitro culture method provided herein.

Moreover, the cell population of the invention may comprise a higher proportion of T-cells with a memory phenotype and/or show in average a more pronounced memory phenotype compared to a control cell population obtained in parallel by the same method except that the control T-cells have not been contacted with an MPC inhibitor.

Thus, the cell population may also refer to a population of T-cells, which comprises, in particular, a higher proportion of T-cells with a memory phenotype, as described herein.

Although various signaling pathways, i.e. PI3K signaling (Eid (2017) Cancer Res. 77(15):4135-4145), rapamycin/mTOR/RICTOR signaling (Araki (2009) Nature 460(7251):108-12; Scholz (2016) EBioMedicine 4:50-61; Li (2012) J Immunol 188(7):3080-7; Zhang (2016) Cell Rep 14(5):1206-1217; Pollizzi (2015) J Clin Invest 125(5):2090-10), and WNT signaling (Muranski (2011) Immunity 35(6):972-85), or AMPK activation (Pearce (2009) Nature 460(7251):103-7) have been implicated in promoting a memory phenotype in human T-cells, the only practical in vitro culture protocol which has been suggested to be useful for the generation and/or maintenance of human T-cells with a memory phenotype, i.e. for the induction of CD62L expression, employs an AKT inhibitor (Klebanoff et al., JCI Insight. 2017 Dec. 7; 2(23):e95103). Furthermore, AKT inhibition, PI3K inhibition or mTOR inhibition may negatively interfere with the activation of T-cells and not provide an optimal yield, as described above. Moreover, the inventors of the present invention have found that the memory-like T-cells generated by in vitro culture with an AKT inhibitor had a reduced capacity of expressing the effector cytokine IFNγ upon restimulation compared to control T-cells. In other words, the human memory-like T-cells according to Klebanoff (2017) loc. cit., seem to have a deficit in an important functional feature associated with memory T-cells, namely the ability to react with an increased amplitude to a reencounter of an antigen.

This further demonstrates that the cell population comprising human T-cells with a memory phenotype obtained by the inventive method provided herein (the human cell product or T-cell product of the invention) is different from the cell populations disclosed in Klebanoff (2017) loc. cit. Furthermore, the inventive cell population provided herein, may be characterized, for example, by an increased percentage of cells expressing CD62L on the membrane, increased trimethylation on the lysine 4 residue of histone 3 (H3K4-3Me), increased acetylation on lysine 27 residue of histone 3 (H3K27-Ac), and/or alterations in the chromatin conformation resulting in more accessible regions (e.g. more than 1000, such as 1633).

Thus, the T-cells of the invention, i.e. in the context of the inventive method and/or cell population provided herein, may have an unaltered or enhanced reactivation capacity, in particular an unaltered or enhanced capacity of producing IFNγ upon restimulation. Preferably, said T-cells are CD8+ T-cells. In particular, the capacity of the herein provided T-cells with a memory phenotype to produce IFNγ upon restimulation may be unaltered or enhanced compared to (seemingly) corresponding T-cells with a memory phenotype that have been obtained by in vitro culture in the absence of an MPC inhibitor. Corresponding T-cells may appear similar to the T-cells of the invention, e.g. for the expression of some markers such as CD62L, and/or may be obtained by a culture method which is similar except for the contacting with an MPC inhibitor. However, said (seemingly) corresponding T-cells may in fact be different in one or more characteristic, e.g. the reactivation capacity as described herein.

In particular, said restimulation may comprise contacting the T-cells with a memory phenotype with Phorbol 12-Myristate 13-Acetate and Ionomycin.

Furthermore, the T-cells with a memory phenotype according to the invention may have a higher expression of activation markers such as CD44, CD71 and/or CD98 compared to T-cells with a memory phenotype that are obtained by in vitro culture in the absence of an MPC inhibitor but in the presence of an AKT inhibitor.

Thus, in one embodiment. the T-cells are not contacted with an AKT inhibitor and/or have not been contacted with an AKT inhibitor.

In a particular embodiment, at least 60%, 70%, 80%, or 90%, preferably at least 70%, e.g. about 75%, of the cells in the inventive cell population provided herein are human CD8+ T-cells with a memory phenotype that express CD62L, in particular, wherein said CD8+ T-cells have not been contacted with an AKT inhibitor.

Furthermore, at least 70% of the human CD8+ T-cells in the inventive cell population provided herein may be T-cells with a memory phenotype that express CD62L, in particular, wherein said CD8+ T-cells have not been contacted with an AKT inhibitor.

In a further particular embodiment,
(a) at least 60%, 70%, 80%, or 90%, preferably at least 70%, e.g. about 75% of the cells in the inventive cell population provided are human CD8+ T-cells,
(b) at least 70% of the human CD8+ T-cells in the inventive cell population provided herein are T-cells with a memory phenotype that express CD62L, and/or the percentage of human CD8+ T-cells that express CD62L and/or the average CD62L expression of the human CD8+ T-cells is greater than in a control cell population comprising human CD8+ T-cells, wherein said control cell population has been obtained by in vitro culture in the absence of an MPC inhibitor.

Furthermore, the T-cells of the invention, i.e. comprised in the cell population of the invention may maintain a memory phenotype in vivo when administered to a subject, as demonstrated in the appended Examples. In particular, the T-cells may efficiently give rise to memory precursor effector T-cells (MPECs) and/or central memory T-cells in vivo, for example in the spleen, when administered to a subject, in particular upon reencounter of the antigen. In particular, said capability or efficiency may be increased compared to T-cells generated by an in vitro culture method which does not employ an MPC inhibitor but is preferably otherwise identical, e.g. a DMSO control, as described herein.

In one embodiment, the T-cells cultured and/or contacted with the MPC inhibitor are human umbilical cord blood (CB) mononuclear cells, human umbilical cord blood (CB) lymphocytes and/or peripheral blood mononuclear cells (PBMC), e.g. PBMCs from umbilical cord blood.

Furthermore, the present invention relates to an in vitro cell culture comprising the inventive cell population provided herein. In particular, said in vitro cell culture may further comprise an MPC inhibitor as described herein. Moreover, said in vitro cell culture may comprise a culture medium described herein in the context of the inventive method.

In one embodiment, the inventive method provided herein comprises a step of transferring cultured T-cells into a subject.

Thus, the cell population of the invention may be used in therapy, i.e. immunotherapy, in particular wherein the cell population or the T-cells comprised in said cell population is/are administered to a patient.

The term “immunotherapy”, as used herein, refers to the treatment of a disease by modulating (activating or suppressing) the immune system. The term “immunotherapy” comprises modulation of the immune system in a subject (in vivo treatment) and/or transferring immune cells which have been modulated during in vitro culture into a subject (cell-based immunotherapy). Preferably, the immunotherapy is a cell-based immunotherapy.

Accordingly, the invention relates to an MPC inhibitor for use in immunotherapy. Preferably, said immunotherapy comprises administering T-cells to a patient, wherein the T-cells have been contacted with the MPC inhibitor during in vitro culture according to the method of the invention, in particular wherein said T-cells have thereby acquired a memory phenotype in vitro.

Accordingly, the invention relates to a population of cells obtained by the method of the invention for use in immunotherapy.

Accordingly, the invention relates to an immunotherapy comprising administering an MPC inhibitor to a patient.

Accordingly, the invention relates to an immunotherapy comprising administering T-cells contacted with an MPC inhibitor to a patient. In particular, the cells are contacted with an MPC inhibitor during in vitro culture.

Accordingly, the invention also relates to a method for generating and/or maintaining T-cells with a memory phenotype in a subject comprising administering an MPC inhibitor to said subject.

In certain embodiments, the immunotherapy comprises T-cells which acquire or have acquired a memory phenotype within a subject, in vitro and/or ex vivo.

In a preferred embodiment, the immunotherapy comprises T-cells which have acquired a memory phenotype in vitro.

In a preferred embodiment, the immunotherapy comprises adoptive cell transfer. Adoptive cell transfer (ACT), as used herein, refers to the transfer of cells into a patient. The cells may have originated from the patient or from another individual. Preferably, the cells have originated from the patient (autologous cells). Preferably, the cells are extracted from the patient, cultured in vitro and returned to the same patient. Alternatively, the cells are isolated and expanded from a donor separate from the patient receiving the cells. The terms “adoptive cell transfer”, “ACT”, “adoptive transfer” and “cells adoptively transferred” are used interchangeably herein. During in vitro culture, the cells may be genetically modified. Preferably, the cells are genetically modified by integrating a TCR or CAR into the genome. The cells may be also modified to inhibit an intrinsic checkpoint. For example, Cytokine-inducible SH2-containing protein (CISH) may be knocked-out or knocked-down in T-cells. Furthermore, the T-cells may be modified to knock out the endogenous TCR in the case of T-cells isolated and expanded from a donor separate from the recipient patient (i.e. in allogeneic T-cells).

In a preferred embodiment, the immunotherapy comprises adoptive transfer of T-cells and/or B-cells which have acquired a memory phenotype in vitro into a patient.

In a preferred embodiment, the immunotherapy is a T-cell therapy. A T-cell therapy, as used herein, refers to the modulation of T-cells in a subject and/or the adoptive transfer of in vitro cultured T-cells into a patient. In a very preferred embodiment, the T-cells therapy comprises adoptive transfer of in vitro cultured T-cells into a patient. Preferably, the cultured and adoptively transferred T-cells are CD8+ T-cells, preferably autologous CD8+ T-cells.

In particular, the T-cells may comprise CD8+ T-cells, the T-cells may be autologous cells, and/or the T-cells may be derived from tumor-infiltrating T-cells.

In one embodiment, the T-cells comprise allogeneic T-cells. For example, the allogeneic T-cells may be, inter alia, from umbilical cord blood. Furthermore, the endogenous TCR of the allogeneic T-cells may be knocked out.

In a preferred embodiment, the immunotherapy is a therapy to treat cancer. An immunotherapy to treat cancer, as used herein, may activate the immune system to contain and/or eliminate cancer cells. The terms “tumor”, “cancer”, “tumor cells” and “cancer cells” are used interchangeably herein and comprise benign and malign tumors as well as single cancer cells, solid tumors, liquid tumors, circulating tumor cells, clusters of cancer cells and metastases. Preferably, the term “cancer” refers to a malign tumor. The invention is not limited to the treatment of a specific type of cancer. Preferably, an MPC inhibitor and/or a population of cells obtained by the method of the invention, may be used for the treatment of melanoma, other solid tumors, and/or hematological malignancies, for example leukemia. The invention may be particularly useful for the treatment of late-stage and/or aggressive cancers, for example metastatic cancer and/or cancer that is resistant to other cancer therapies.

In a preferred embodiment, the cancer is resistant to chemotherapy, targeted therapy and/or antibody-mediated immunotherapy.

Chemotherapy, as used herein, refers to a type of cancer treatment that uses one or more anti-cancer drugs (chemotherapeutic agents) as part of a standardized chemotherapy regimen. Typically, a chemotherapeutic agent inhibits cell division. Typical chemotherapeutic agents are, for example, but not limited to, Chlorambucil, Valrubicin, Abraxane, Vorinostat, Irinotecan, Etoposide, Bortezomib, Vemurafenib, Fluorouracil, Actinomycin, Oxaliplatin, Tretinoin and Vinblastine.

Targeted therapy, as used herein, refers to blocking the growth of cancer cells by interfering with specific targeted molecules needed for carcinogenesis and tumor growth. Typically, compounds used for targeted therapy are small molecules and/or monoclonal antibodies. Typical small molecules for targeted therapy are, for example, but not limited to Imatinib, Erlotinib, Vemurafenib, Everolimus, Obatoclax, Crizotinib, Sunitinib. Typical monoclonal antibodies for targeted therapy are, for example, but not limited to Rituximab, Trastuzumab, Bevacizumab and Cetuximab.

In some cases, a small molecule can be classified both for use in chemotherapy and targeted therapy. In contrast, a small molecule which is known for use in chemotherapy and/or targeted therapy cannot be predicted to have an effect in immunotherapy. Chemotherapy and/or targeted therapy usually act directly on cancer cells, whereas immunotherapy acts primarily on immune cells. Monoclonal antibodies, however, may be classified both for use in targeted therapy and immunotherapy, because they may, in contrast to small molecules, both interfere with a cancer-associated molecule and stimulate the immune system.

The term “antibody mediated immunotherapy”, as used herein, refers to monoclonal antibodies which modulate and/or stimulate the immune system, for example through Antibody-dependent cell-mediated cytotoxicity (ADCC), the complement system and/or blocking immunosuppressive mechanisms (checkpoints) such as the PD-1/PD-L1 interaction. Typical monoclonal antibodies for targeted therapy are, for example, but not limited to Alemtuzumab, Durvalumab, Nivolumab, Pembrolizumab, Trastuzumab, Pertuzumab, Monalizumab and Rituximab.

In a preferred embodiment, the cancer comprises metastases.

In some embodiments, the cell population of the invention and/or T-cells with a memory phenotype according to the invention is/are administered to a patient in combination with an additional anti-cancer drug, preferably a checkpoint inhibitor, as described herein, i.e. in the context of the composition comprising an MPC inhibitor. The additional anti-cancer drug may be administered before, concomitantly and/or after administration of the cell population/T-cells of the invention.

In one embodiment, the immunotherapy comprises administering an MPC inhibitor to a subject. Preferably, the MPC inhibitor is administered to a subject for the treatment of cancer.

The invention also relates to a composition comprising an MPC inhibitor for use in immunotherapy.

In one embodiment, a composition for use in the treatment of cancer comprises at least one additional anti-cancer drug. Said anti-cancer drug(s) may be selected from a chemotherapeutic agent, an agent for targeted therapy and/or a monoclonal antibody for antibody mediated immunotherapy, as described herein.

In a preferred embodiment, the composition for use in the treatment of cancer further comprises a checkpoint inhibitor, for example a molecule which targets CTLA4, PD-1, and/or PD-L1, such as, but not limited to, Ipilimumab, Pembrolizumab, Nivolumab, Atezolizumab and Avelumab.

In some embodiments, the immunotherapy is a therapy to treat a chronic viral infection. Preferably, the chronic viral infection is HIV. Without being bound by theory, a long-lasting pool of HIV specific T-cells with a memory phenotype may increase the chance of eradicating HIV infected cells.

In some embodiments, the immunotherapy is a therapy to treat an autoimmune disease. Autoimmune diseases are, for example, but not limited to celiac disease, diabetes mellitus type 1, Graves' disease, inflammatory bowel disease, multiple sclerosis, psoriasis, rheumatoid arthritis, and systemic lupus erythematosus. Preferably, the cells used for treating an autoimmune disease are regulatory T-cells with a memory phenotype. Such long-lasting regulatory T-cells may be used to protect a patient against aberrant immune responses; Rosenblum et al.; Nat Rev Immunol. 2016 February; 16(2):90-101. Additionally, cytotoxic T-cells and/or helper T-cells with a memory phenotype and/or antibodies derived from B-cells with a memory phenotype may recognize specific T-cells which contribute to an autoimmune reaction and cause or contribute to the elimination of the latter.

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

The invention is also characterized by the following figures, figure legends and the following non-limiting examples.

### BRIEF DESCRIPTION OF THE FIGURES

FIG. 1: Treating CD8 T cells with a small molecule inhibiting the mitochondrial pyruvate carrier (UK5099, MPCi) results in enhanced memory characteristics that are maintained during Listeria infection in vivo.

(A) Schematic representation of the in vitro mouse CD8 T cell activation and treatment. (B) FACS analysis at day 7 showing CD62L histograms of OT1 cells activated in the presence of MPCi (UK5099) or the solvent DMSO. (C) RNAseq data revealing the fold change in gene expression in mouse CD8 T cells upon UK5099 treatment at day 7 as compared to DMSO (D) Quantification of the percentage of mouse CD8 T cells expressing low or high levels of CD62L as measured by flow cytometry. (E) Schematic representation of the ex vivo activation and treatment of human cord blood PBMCs. (F-G) Representative FACS histogram (F) and analysis (G) at day 11 showing CD62L expression in MPCi-treated human CD8 T-cells versus DMSO control-treated cells. (H) Schematic representation of Listeria-Ova infection. 100,000 OT-1 cells cultured in the presence of MPCi or DMSO were transferred into naïve recipients followed by 2000 cfu Listeria-Ova infection. (I) Percentage of transferred cells (CD45.1+) out of the total CD8+ T cell population in the blood upon transfer of MPCi-treated T-cells. (J) Representative plots and quantification of flow cytometry data of KLRG1-negative and CD127-positive cells in the CD8+CD45.1+ population in the blood at day 7 post-infection. (K) Representative plots of flow cytometry data of CD44 and CD62L double positive cells in the CD8+CD45.1+ population in the blood at day 28 post-infection. (L-N) T cell persistence (L), MPEC at d7 (M) and central memory T cells at d28 post infection (N) in mice receiving adoptively transferred WT or MPC1 KO T cells. *p<0.05 versus DMSO or WT. Graphs show mean±Standar Error of Mean (SEM).

FIG. 2: MPCi-treated CD8 T cells show enhanced anti-tumoral activity. (A) Schematic representation of the tumor experiment. 1 day after a low dose (5Gy) whole body irradiation, 100,000 OT-1 cells, either cultured with the small molecule or DMSO, were transferred into B16-OVA tumor-bearing mice. Mice were subsequently vaccinated s.c. with CpG and OVA peptide. (B) B16-OVA tumor growth in mice upon transfer of MPCi- or DMSO treated cells, or in untreated mice (PBS). (C) Percentage of CD44 and CD62L double positive T cells out of the transferred CD8+CD45.1+ population in the spleen. (D) Percentage of PD-1 positive T cells out of the transferred CD8+CD45.1+ population in the tumor. (E) The single-cell suspension of tumors was re-stimulated with SIINFEKL peptide for 4 h. IFNγ and TNFα production was measured by flow cytometry. *p<0.05 versus DMSO. Graphs show mean±SEM.

FIG. 3: Epigenetic mechanisms underlie the enhanced memory characteristics of MPCi-treated CD8 T cells. (A) Total acetyl-CoA levels measured by mass spectrometry. (B) Percentage of acetyl incorporation by carbons derived from 13C-glucose or 13C-glutamine into the total acetyl-CoA pool. (C) Western blot showing trimethylation of lysine 4 (H3K4-3Me) and acetylation of lysine 27 (H3K27-Ac) on histone 3 upon UK5099 treatment. (D) Visualization of the number of genes that are associated with more open or less open chromatin regions upon MPCi treatment. (E) Example of ATAC-seq traces in the gene loci of Sell, Tcf7 and Ccr7 of OT1 T cells treated with UK5099 or DMSO. *p<0.05 versus DMSO. Graphs show mean±SEM.

FIG. 4: UK5099 as adjuvant drives a memory phenotype in T cells upon vaccination and protects against tumor growth. (A) Schematic representation of the vaccination experiment. UK5099 (MPCi) was given s.c. at 0.5 mg/mouse at d0 and d2. (B) Quantification of flow cytometry data of KLRG1-negative and CD127-positive MPECs in the CD8+CD45.1+ population in the blood at day 14 post-vaccination. (C-D) B16-OVA tumor growth (C) and weight (D). *p<0.05 versus DMSO. Graphs show mean±SEM.

FIG. 5: Phenotypic analysis of mouse and human CD4 T cell activation upon MPCi treatment. (A) Schematic representation of mouse CD4 T cell activation and treatment. (B and C) Histogram representing flow cytometry data on CD62L expression in CD4 T cells upon treatment with DMSO or MPC inhibitor (UK5099) (B), and the quantification thereof (C). (D) Quantification of flow cytometry analysis at day 11 post-activation of human cord blood PBMCs showing the percentage of CD62L+CD4 T cells in the CD3-positive/CD8-negative T cell population. *p<0.05, compared to DMSO. Graph shows mean±SEM.

FIG. 6: MPC inhibition during the production of murine CAR T cells improves their memory phenotype and antitumor function upon adoptive cell transfer therapy. (A) Experimental design. (B) Tumor growth curve. (C) Number of Her2-CAR T cells per tumor-draining lymph node. (D) Number of Her2-CAR T cells per spleen. (E) Percentage of TCF1-positive cells out of Her2-CAR T-cells in the spleen. (F) Number of Her2-CAR T cells per mg of tumor. (G) Percentage of TCF1-positive cells out of Her2-CAR T cells in the tumor. (H and I) Percentages of progenitor-exhausted T cells (H) and terminally exhausted T cells (I) out of Her2-CAR T cells in the tumor. (J) Percentage of exhaustion marker (PD1 and TIM3)-expressing Her2-CAR T cells in the tumor. Pooled data from 2 independent experiments. ns: non significant, *p<0.05, **p>0.01, ***p<0.001. Data represents mean±s.e.m.

FIG. 7: MPC inhibition during the activation and culture of adult human T cells induces a memory phenotype. (A) Experimental design. PBMCs were isolated from adult healthy volunteers and activated with anti-CD3/CD28 beads in the presence of DMSO or UK5099 (25 μM). Beads were removed at day 5 and T cell phenotype was analysed at day 9. (B) Percentage of CD8 T cells expressing the memory marker CD62L. (C) The mean fluorescent intensity of CD62L in the respective positive population. (D) Percentage of CD8 T cells expressing markers that allows their identification as stem cell memory T cells (TSCM). (E-F) Mitochonrial mass (identified by MitoGreen staining, E) and mitochondrial membrane potential (TMRM, F), measured by flow cytometry and expressed as fold change as compared to DMSO. (G) Mitochondrial membrane potential normalized for the mitochondrial mass, expressed as the ratio of MitoGreen over TMRM, fold change as compared to DMSO. Pooled data from 2 independant experiments. ns: non-significant, *p<0.05, **p>0.01, ***p<0.001. Data represents mean±s.e.m.

### EXAMPLES

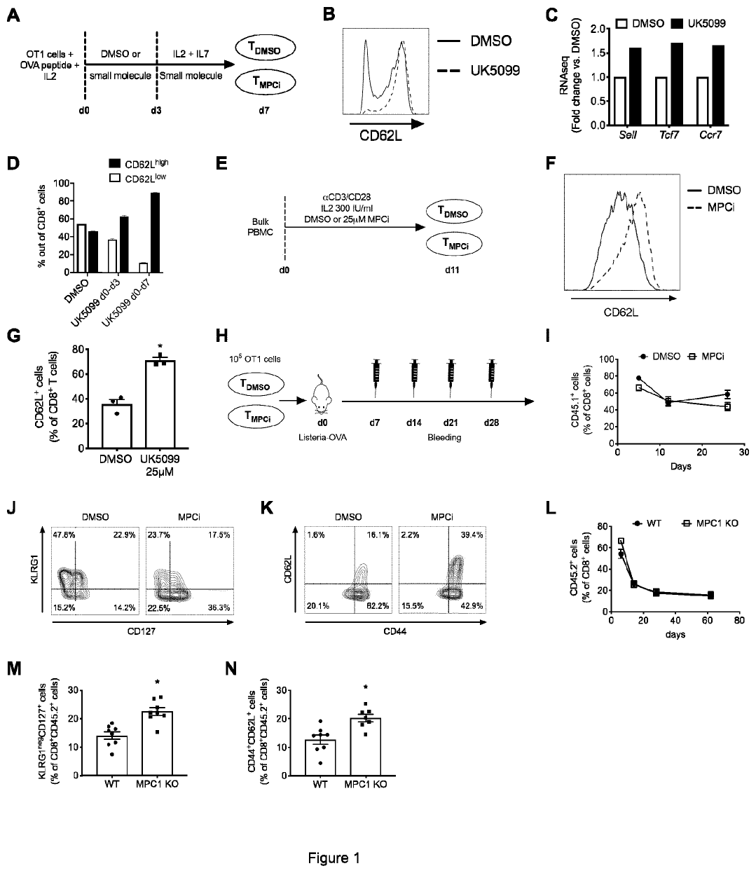

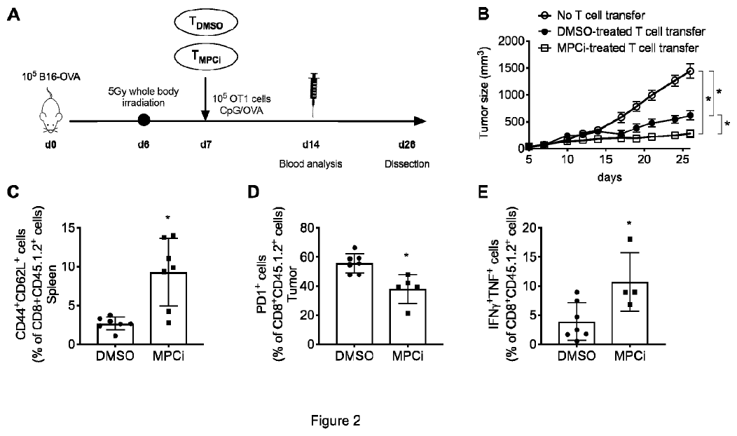

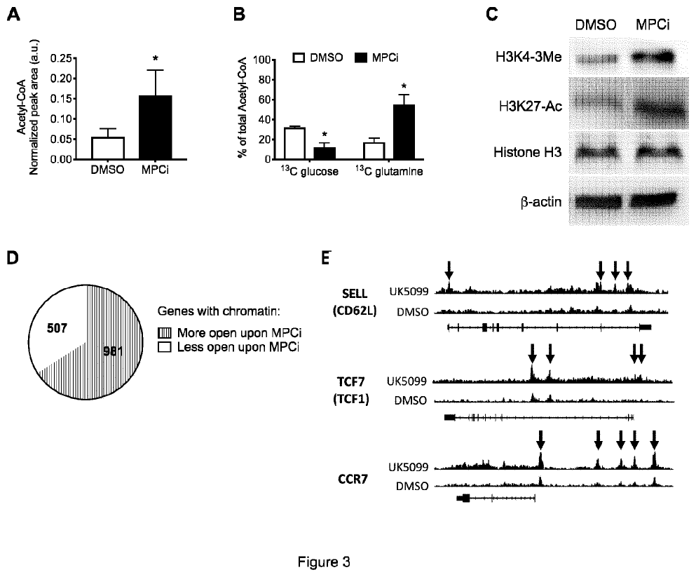

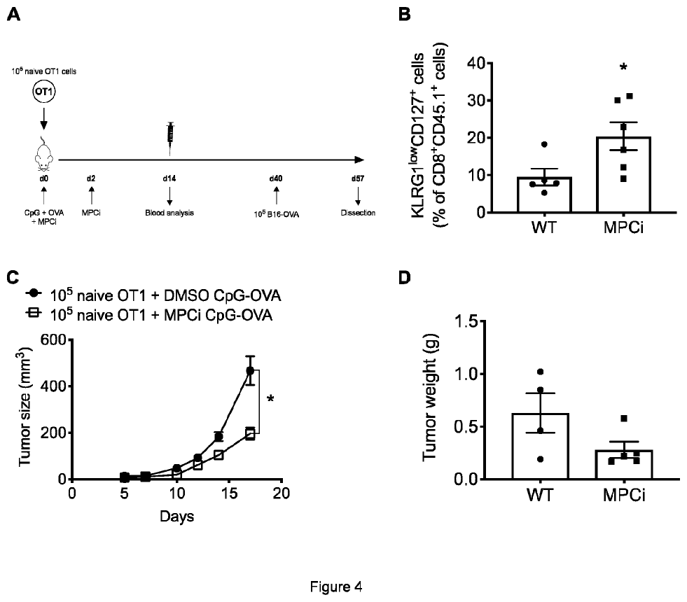

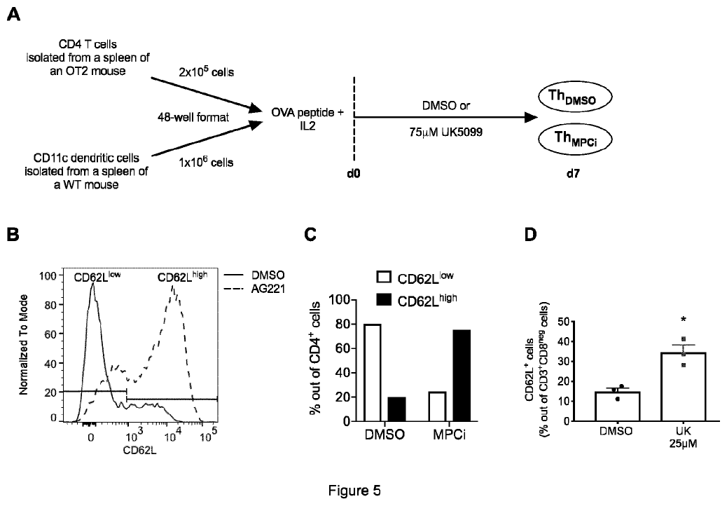

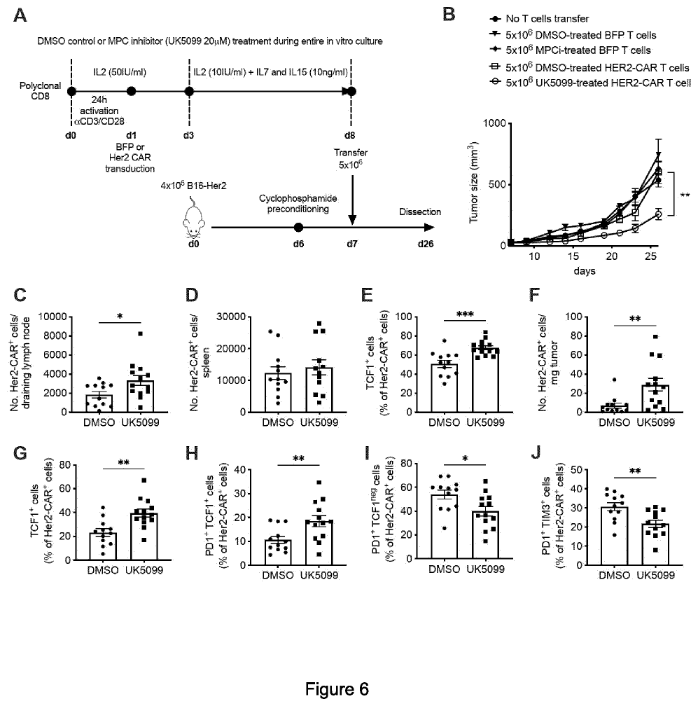

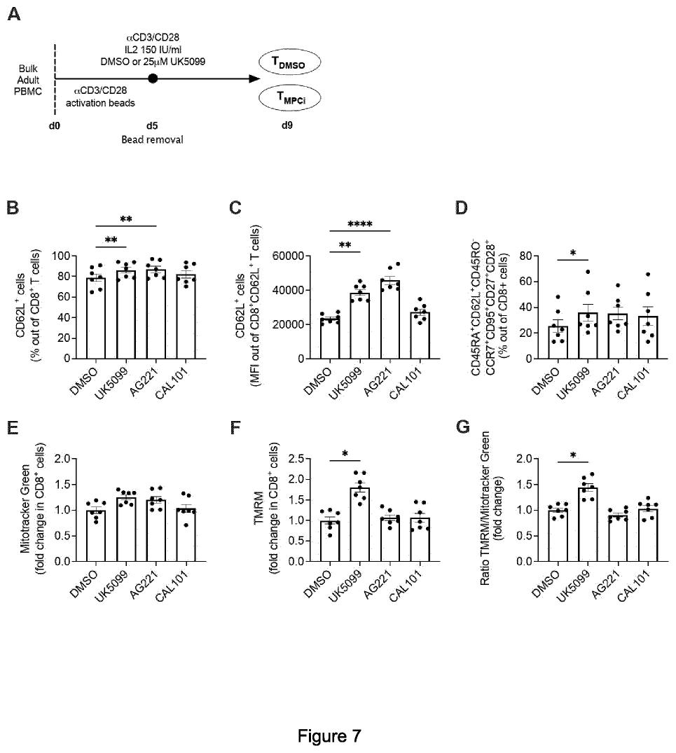

To evaluate the effects of MPC inhibition on T cell differentiation, OT1 splenocytes were cultured in the presence of 1 μg/ml ovalbumin-derived N4 (SIINFEKL) peptide, 100 IU/ml recombinant human IL-2 (rhIL-2) and the MPC inhibitor (MPCi) UK5099 at 75 μM or control DMSO for 3 days. The inhibitor or DMSO was then washed away and the cells were cultured for 4 more days in the presence of 100 IU/ml IL-2 and 10 ng/ml rhIL-7 (FIG. 1A). When analyzing the surface expression of the central memory marker CD62L by flow cytometry on day 7, we could observe a strong increase in cells treated with MPCi compared to DMSO (FIG. 1i). Furthermore, RNA sequencing revealed that Sell (CD62L) expression was at least 1.5-fold increased at day 7 in cells treated with MPCi, as well as other memory markers Tcf7 and Ccr7 (FIG. 1C). Thus, the increase in the memory markers upon MPCi in vitro treatment occurs at the transcriptional level. Of note, also continuing MPCi treatment between d3 and d7 induced an upregulation of surface CD62L expression at day 7, even more pronounced than a d0-d3 treatment only (FIG. 1D). This indicates that the memory induction induced between d0 and d3 is stable until day 7, but a continuous treatment with MPCi is inducing the highest CD62L expression.

Important for the clinical translation is to prove whether an improved memory T cell differentiation can also be obtained in human CD8 T cells. To most closely mimic the experimental conditions that were successful by using naïve mouse OT1 CD8 T cells, it was tested whether the MPCi on the most naïve human CD8+ T cells can be obtained, i.e. from umbilical cord blood (FIG. 1E). Importantly, in 3 different donors a significant increase in CD62L expression with MPCi was observed (FIGS. 1F and 1G).

In order to evaluate whether this memory phenotype is maintained in vivo, 10⁵ MPCi- or DMSO-treated OT1 cells were intravenously injected in healthy mice. Twenty-four hours after cell transfer, the mice were infected with a sub-lethal dose of ovalbumine-expressing Listeria monocytogenes (LM-OVA, 2000 CFU i.v.) (FIG. 1H). Weekly bleeding of the mice did not reveal significant differences in the frequency of the transferred cells during the peak of the immune response, nor during the contraction phase (FIG. 1I). At the peak of the immune response, day 7 post-LM-OVA, the frequency of memory precursor cells (CD127+, KLRG1low) was significantly increased in OT1 cells pretreated with the MPCi (FIG. 1J). Consequently, 28 days after LM-OVA infection, during the memory phase of the immune reaction, the proportion of central memory T cells (CD44+CD62L+) among the transferred cells in the blood was larger in mice that received cells pre-treated with MPCi (FIG. 1K). Importantly, the effects of the MPC inhibitor were target specific, since an increase in memory T cell differentiation could also be observed when MPC1 KO T cells were transferred in this Listeria model (FIG. 1L-N). In summary, MPCi-treated cells display a consistent memory phenotype upon in vivo transfer in an infection model.

Adoptive Cell Transfer Therapy with MPCi-Treated CD8 T Cells is Better Able to Control Tumor Growth in a Mouse Melanoma Model

To assess the functional capacity of the generated memory T cells, the MPCi-treated cells were further tested in a mouse model of melanoma. Briefly, 10⁵ ovalbumin-expressing B16 melanoma cells were injected subcutaneously in 6-week-old mice. At day 6 post-engraftment, when a palpable tumor was present, mice were irradiated with 5Gy. The next day 10⁵ MPCi- or DMSO-treated OT1 cells were intravenously injected, followed by a subcutaneous vaccination of 50 μg CpG and 10 μg SIINFEKL (FIG. 2A). It has been shown before that memory CD8+ T cells are more potent in controlling tumor growth, and indeed, B16 tumor growth was strongly reduced in mice that received OT1 CD8+ T cells pre-treated with MPCi as compared to DMSO (FIG. 2B). However, upon dissection, the percentage of transferred cells out of total CD8+ T cells was not strikingly different in the spleen and tumor in MPCi mice (data not shown). Confirming the Listeria data, the proportion of central memory CD8+ T cells in the spleen was increased upon MPCi treatment (FIG. 2C). Interestingly, MPCi-treated T cells infiltrating the tumor had a significantly decreased expression of the immunosuppressive molecule PD-1 (FIG. 2D). When restimulating the single cell population of the tumor in vitro with the OVA peptide SIINFEKL, OT1 T cells treated with MPCi responded much better, with a higher proportion of cells being double positive for IFNγ and TNF (FIG. 2E).

### An Epigenetic Mechanism Might be Responsible for the Durable In Vivo Memory Responses Upon In Vitro Metabolic Intervention

Mechanistically, it was hypothesized by the inventors that epigenetic processes might underlie the observed long-term memory phenotype and anti-tumor activity upon metabolic intervention with MPCi. Several metabolites were differentially abundant, but most strikingly was a strong increase in acetyl-CoA upon MPCi treatment (FIG. 3A). When trying to determine the carbon source of this increased acetyl-CoA, it was observed that 13C-labeled glucose is as expected incorporating less upon MPCi treatment. Instead 13C-glutamine incorporated dramatically more into acetyl-CoA after mitochondrial pyruvate carrier inhibition (FIG. 3B). Since acetyl-CoA is the acetyl donor for the acetylation of proteins, amongst which also histones, histone post-translational modifications were examined that are known to make chromatin accessible to transcription factors, a characteristic of memory T cells. Interestingly, an increase in acetylation of lysine 27 on histone 3 was observed, but also in trimethylation of lysine 4, upon UK5099 treatment (FIG. 3C). These are marks known to open chromatin and make it accessible for transcription factor docking. Indeed, ATAC-seq (Assay for Transposase-Accessible Chromatin using sequencing) revealed that 981 genes were associated with more accessible chromatin upon MPCi treatment (FIG. 3D). Amongst those were Sell, Tcf7 and Ccr7, thereby correlating accessible chromatin with increased mRNA copies (FIG. 3E and see above).

Together, these results show that short-term metabolic intervention can lead to enhanced memory characteristics and improved anti-tumor control, likely by an epigenetic mechanism.

### An MPC Inhibitor as Vaccine Adjuvant Induces a Better Memory CD8 T Cell Development and Protects Against Subsequent Tumor Challenge

One of the main goals of preventive and therapeutic vaccination in the context of pathogen infection and cancer is the establishment of a potent memory CD8 T cell pool. Since it was observed that MPC inhibition during in vitro priming of antigen-specific CD8 T cells increases their memory formation, it was investigated whether the MPCi could be used as a vaccine adjuvant. 10⁵ naïve OT1 CD8 T cells were transformed in healthy mice and the mice were subsequently vaccinated by a subcutaneous injection of 50 μg CpG and 10 μg SIINFEKL containing additionally 0.5 mg UK5099 or and equal volume of DMSO as control. 48 hours later, the mice received another subcutaneous injection of 0.5 mg UK5099 or and equal volume of DMSO (FIG. 4A). 14 days after vaccination, an increased proportion of the transferred OT1 CD8 T cells was observed that formed memory precursor cells (FIG. 4B). It was investigated whether this would render them better prepared for a subsequent antigen challenge, and injected 10⁵ ovalbumine-expressing B16 melanoma cells s.c. in the flank of the mice at 40 days post-vaccination (FIG. 4A). Strikingly, the mice that received the vaccine formulated with the MPCi were much better in controlling the tumor growth (FIGS. 4C and 4D). This preliminary experiment indicates that MPC inhibition during the priming phase of in vivo vaccination might induce a stronger memory pool formation and protect against future disease.

### Mice and Tumor Lines

Mice were maintained in the animal facility of the University of Lausanne. OT1 mice were bred on site and C57BL/6 (B6) mice were obtained from ENVIGO. MPC1flox/flox mice were generated by Dr. Jared Rutter and intercrossed in our facility with CD4.CRE mice and OT1 mice. B16-Ova melanoma tumor cell line was generated previously in the laboratory. All experiments were performed in accordance with Swiss federal regulations and procedures approved by veterinary authority of the Canton de Vaud.

### Mouse Splenocyte Culture

OT1 splenocytes were cultured for 3 days at a concentration of 106 cells per mL in RPMI medium (Gibco 61870-01) supplemented with 10% FBS, (Gibco 10270-106), 1% Penicillin/Streptomycin (Gibco 15070-063), 50 μM β-mercaptoethanol, 1% HEPES (Gibco 15630-080), 1× Non-essential amino acids (Gibco 11140-035), 1% L-glutamine (Gibco 25030-081), 1 mM Sodium Pyruvate (Gibco 11360-039). Cells were additionally supplemented with hIL-2 100 U/ml (Glaxo-IMB), ovalbumin N4 peptide (SIINFEKL) 1 μg/ml and either with 75 μM UK5099 (Sigma Aldrich) or with their solvent DMSO as a control. At day 3, splenocytes were collected, washed and split, and cells were cultured for 4 additional days with 100 U/ml hIL-2 and hIL-7 (Peprotech 200-07) supplemented either with 75 μM UK5099 (Sigma Aldrich) or DMSO. At day 7, flow cytometry analyses were performed for surface marker expression.

Naïve CD4 T cells were isolated by negative selection (Stem Cell Technologies) from the spleen of OT2 mice. Dendritic cells were isolated from spleens of C57BL/6 mice by CD11c positive selection (Stem Cell Technologies). 2×10⁵ CD4 T cells were co-cultured with 1×10⁶ dendritic cells in RPMI medium supplemented with 10% FBS, 1% Penicillin/Streptomycin, 50 μM β-mercaptoethanol, 1% HEPES, 1× Non-essential amino acids (Gibco 11140-035), 2 mM L-glutamine and 1 mM Sodium Pyruvate. T cells were activated by adding 1 μg/ml Ovalbumine peptide (323-339, ISQAVHAAHAEINEAGR) and 100 IU/ml rhIL-2, in the presence of 75 μM UK5099 or DMSO. Cells were split on day 4 and re-cultured in fresh medium containing 100 IU/ml rhIL-2, in the presence of 75 μM UK5099 or DMSO.

### Human Cord Blood PBMC Culture

Peripheral blood mononuclear cells were isolated from fresh umbilical vein cord blood on a Percoll gradient. PBMCs were then cultured in RPMI supplemented with 10% human serum. 10⁴ PBMC's were seeded per well in a round bottom 96-well plate and activated with anti-CD3/CD28 beads at a 1:2 cell:bead ratio and 300 U/ml rhIL-2, in the presence of 25 μM UK5099 or DMSO. Cells were regularly split and CD62L expression was determined by flow cytometry on day 11.

### Adoptive Cell Transfer

Activated CD45.1+OT-1 splenocytes were culture in vitro for 7 days as described above, collected and purified on a Ficoll gradient, allowing to separate dead and live splenocytes. Live splenocytes were counted with Trypan blue stain 0.4%. 100′000 live splenocytes were transferred into CD45.2+ host mice by tail vein injection.

### In Vivo Listeria-Ova Infection Model

Recombinant bacteria Listeria monocytogenes deficient for actA and expressing the ovalbumin (Ova) peptide SIINFEKL were expanded and tittered. Optical density measured with a spectrophotometer was used to determine bacterial concentration and 2000 CFU were administered in each mouse by tail vein injection, 4 hours after adoptive cell transfer. Blood samples were collected and processed for surface markers and cytokine production analyses.

### Melanoma Tumor Model

B16-OVA cells were cultured in DMEM (GIBCO) with 10% FBS and 1% P/S before their subcutaneous injection into the mouse flank. Each mouse received 100′000 cells in a volume of 200 μl of PBS. 6 days after B16-OVA cells injection, tumors were measured, mice were randomized and lymphodepleted by irradiation (5 Gray). 7 days after B16-Ova injection, mice were adoptively transferred with the ACT protocol described previously. Following the ACT, mice received a vaccination of CpG (50 μg/mouse) and N4 Ova peptide (10 μg/mouse) diluted in PBS to obtain a total volume of 100 μl/mouse, injected subcutaneously at the tail base. Tumors were measured every 2 days and the tumor volume was calculated according to the formula: V=π×[d2×D]/6, where d is the minor tumor axis and D is the major tumor axis. At day 26 days post-tumor engraftment, tumors and spleens were dissected were then stained for flow cytometry analyses.

### Vaccination Model

Naïve CD8 T cells were isolated from OT1 spleens (negative selection, Stem Cell Technologies). 1×10⁵ T cells were i.v. transferred in WT C57BL/6 mice, followed by a subcutaneous injection of the vaccine, consisting of 10 μg SIINFEKL peptide, 50 μg CpG and 0.5 mg UK5099 or DMSO diluted in a total of 100 μl PBS per mouse. 2 days post-vaccination, mice received a second dose of s.c. 0.5 mg UK5099 or DMSO. After 2 weeks, blood was collected from the tail vein and analyzed by flow cytometry. 40 days post-vaccination, mice were challenged by an s.c. injection of 1×105 SIINFEKL-expressing B16 melanoma cells. 17 days later, mice were sacrificed and the tumor was dissected.

### Flow Cytometry

Cells were incubated in a live/dead stain (Fixable aqua dead cell stain kit, Invitrogen) followed by an incubation with different antibody panels.

Antibodies used for in vitro surface analyses were: CD8-PE-texas-Red, CD62L-FITC, CD127-PE, CD27-PerCP-Cy5.5, CD71-PE-Cy7, CD44-APC-Cy7, CD98-APC, CD25-Pacific blue (BD Pharmingen and eBioscience, San Diego, CA, USA).

Antibody panels for blood analyses contained: CD8-PE-texas-Red, CD45.1-Pacific blue, CD45.2-FITC, CD127-PE, KLRG1-PE-Cy7, CD62L-APC, CD44-APC-Cy7, and CD27-PerCP-Cy5.5.

Antibody panel for intra-tumoral T cells and splenocytic T cells was: CD8-PE-texas-Red, CD45.1-Pacific blue, CD45.2-FITC, CD62L-PE-Cy7, CD44-APC-Cy7, PD1-APC, Lag3-PE, and CD127-FITC.

Antibody panel for cytokine detection contained: CD8-PE-texas-Red, CD45.1-FITC, CD45.2-APC, TNFa-Pacific blue, IFNg-PerCP-Cy5.5, IL-2-PE.

Cells were acquired on LSR-II flow cytometers from the flow cytometry facility of UNIL and data were analyzed with FlowJoTM10 software. Antibodies were purchased from BD Phamingen (San Diego, CA, USA), eBioscience (San Diego, CA, USA, and Biolegend (San Diego, CA, USA).

### Metabolomics

OT1 splenocytes were activated and cultured as described above. After 66 hours, the cells were collected and the medium was replaced with glucose- and glutamine-free RPMI, supplemented with 10% dialyzed FBS, 1% P/S, 10 mM HEPES, 50 μM β-mercaptoethanol and either 11 mM normal glucose or 11 mM U-¹³C6-glucose (Cambridge Isotopes) with respectively 4 mM ¹³C5-glutamine (Cambridge Isotopes or 4 mM normal L-glutamine. After 6 hours labelling at 37° C., cells were collected and lysed in Methanol. Next Chloroform was added, and after centrifugation, the fraction containing the polar metabolites was collected and dried on a SpeedVac. Samples were then resuspended and loaded on a mass spectrometer. The spectra of AcetylCoA were analyzed with the M+0 representing the unlabeled fraction, and the M+2 representing the Acetyl group derived from the ¹³C-labelled substrate (glucose or glutamine).

### RNA Sequencing and Analysis

mRNA was extracted from OT1 T cells on day 3 of culture (Qiagen RNeasy kit) and sequenced on the Illumina HiSeq platform. Purity-filtered reads were adapters and quality trimmed with Cutadapt (v. 1.8, Martin 2011). Reads matching to ribosomal RNA sequences were removed with fastq_screen (v. 0.9.3). Remaining reads were further filtered for low complexity with reaper (v. 15-065, Davis et al. 2013). Reads were aligned against Mus musculus.GRCm38.86 genome using STAR (v. 2.5.2b, Dobin et al. 2013). The number of read counts per gene locus was summarized with htseq-count (v. 0.6.1, Anders et al. 2014) using Mus musculus.GRCm38.86gene annotation. Quality of the RNA-seq data alignment was assessed using RSeQC (v. 2.3.7, Wang et al. 2012). Reads were also aligned to the Mus musculus.GRCm38.86 transcriptome using STAR (v. 2.5.2b, Dobin et al. 2013) and the estimation of the isoforms abundance was computed using RSEM (v. 1.2.31, Li and Dewey 2011). Statistical analysis was performed for genes in R (R version 3.4.0). An analysis with all genes, including mitochondria, was done. Genes with less than one count per million in all samples were filtered out. Library sizes were scaled using TMM normalization (EdgeR package version 3.14.0; Robinson et al. 2010) and log-transformed with EdgeR cpm function.

Differential expression was computed with limma (Ritchie et al. 2015) by fitting the samples into a linear model using all conditions as factors and correcting for batch effect by introducing factors for the replicates (=paired analysis).

### ATAC Sequencing

After 3 days of OT1 CD8 T cell culture, 5×10⁴ cells were collected and transposed as described before (Buenrostro et al, Transposition of native chromatin for multimodal regulatory analysis and personal epigenomics. Nat Methods. 2013 December; 10(12): 1213-1218.). Amplified transposed fragments were sequenced on an Illumina HiSeq platform.

Computations of the analysis were performed at the Vital-IT Center for high-performance computing of the SIB Swiss Institute of Bioinformatics. Sequencing reads contained in fastq files were aligned to the Mouse mm10 reference genome using Bowtie2 v.2.3.4.1, and alignment files were manipulated with samtools v.1.8. Peak calling was performed with Macs2 v.2.1.1. Differential peak analysis was performed in R v.3.5.1 with package DiffBind v.2.10.0. Genomic feature annotation was performed using CHIPpeakAnno v.3.16.0 and rGREAT v.1.14.0.

### Western Blot

Cells were lysed in RIPA lysis buffer (50 mM TrisHCl pH8, 150 mM NaCl, 1% Triton X 100, 0.5% Sodium deoxycholate, 0.1% SDS) and Halt protease/phosphatase cocktail inhibitors (Roche) and denatured by heat. Proteins were quantified by BCA protein assay kit (Thermo Scientific). Proteins were separated on 12.5% polyacrylamide gradient gels and transferred onto nitrocellulose membranes 0.2 μm (Biorad). Non-specific binding sites were blocked in milk 5% and membranes were incubated with primary antibodies (Cell Signaling). Membranes were then incubated with HRP-labeled secondary antibodies anti-rabbit (1:1000) and anti-mouse (1:10′000) (Santa Cruz Biotechnology), and blots were visualized by chemiluminescence with ECL and femto reagents (Super Signal West, Thermo Scientific).

### Statistical Analyses

Statistical analyses were performed in GraphPad Prism 7 software using different statistical tests indicated for each experiment. Results are shown in mean±SEM and P<0.05 was considered statistically significant.

### Mouse and Human CD4 Data

An experiment has been performed on mouse T cells in order to evaluate if MPC inhibition also promotes memory marker expression in CD4 T cells. As indicated in the experimental scheme (FIG. 5A), CD4 T cells from OT2 mice were activated by co-culture with dendritic cells. OT2 mice are transgenic for an D DTCR recognizing specifically a chicken ovalbumine peptide 323-339 associated with the mouse MHC class II molecule I-Ab. We activated OT2 CD4 T cells by co-culture with dendritic cells loaded with 1p g/ml of the ovalbumine peptide in the presence of 100 IU/ml recombinant human IL2 in the presence of 75 μM UK5099 or DMSO control. After 4 days, cells were collected, washed and split, and brought back in culture with 100 IU/ml rhIL2 and 75 μM UK5099 or DMSO control. At day 7, CD62L expression was determined by flow cytometry and showed a strong increase in the CD62L-positive population upon IDH2i treatment (FIGS. 5B and 5C).

CD62L expression in human CD4 T cells was also analyzed. Human cord blood PBMCs were cultured and activated as described before, in the presence of 25p M UK5099 or DMSO control. About 98% of the cells in culture on day 11 are CD3-positive. Since CD3 is exclusively expressed on CD4 and CD8 T cells, we can deduct that all CD3-positive, CD8-negative must be CD4 T cells. When analyzing the CD3-pos/CD8-neg population we observed that CD62L expression is increased upon MPCi treatment (FIG. 5D).

### Measurement of MPC Inhibitory Activity

Potential drug interactions with the MPC were assessed using a bioluminescence energy transfer (BRET)-based MPC activity system called reporter sensitive to pyruvate (RESPYR). Briefly, described MPC2-RLuc8 and MPC1-Venus fusion proteins were stably expressed in HEK293 cells using lentiviral transduction. Cells were plated in white 96-well plates 48 hr before recording. Cells were washed with PBS-CM (PBS supplemented with 1 mM CaCl₂) and 0.5 mM MgCl2), and readings were performed 5 min after addition of 5 mM coelenterazine h substrate (Invitrogen). Signals were detected with two filter settings (R-Luc8 filter, 460±40 nm; and Venus filter, 528±20 nm) at 37 C using the Synergy 2 plate reader (Biotek). The BRET value was defined as the difference between the emission at 528 nm/460 nm of co-transfected R-Luc8 and Venus fusion proteins (MPC2-R+MPC1-V) and the emission at 528 nm/460 nm of the R-Luc8 fusion protein alone (MPC2-R). Results were expressed in milliBRET units (mBU). MPC inhibitors are added after baseline readings in the absence of pyruvate.

MPC Inhibition During the Production of Murine CAR T Cells Improves their Memory Phenotype and Antitumor Function Upon Adoptive Cell Transfer Therapy.

The data obtained in mice made use of CD8 T cells isolated from transgenic OT1 mice, which were designed to express one unique T cell receptor recognizing a peptide sequence of the chicken ovalbumin protein (SIINFEKL) when presented on MHC class I molecules. The inventors were able to show that OT1 T cells, activated and cultured in vitro in the presence of an MPC inhibitor, displayed improved anti-tumor activity when transferred in mice bearing melanoma B16 tumor that were genetically engineered to express the SIINFEKL peptide.

It was intended to extend this data by evaluating if this method can also be applied during the production of mouse CAR T cells. Therefore, a CAR construct recognizing human HER2 was used, an oncogene frequently involved in human breast cancer, containing a 4-1BB costimulatory domain.

For retrovirus preparation, a modified protocol from Tschumi et al, J Immunother Cancer. 2018 Jul. 13; 6(1):71 was used.

For each retroviral preparation, 8×10⁶ Phoenix ECO cells (ATCC, CRL-3214) were plated in a T150 tissue culture flask in RPMI medium supplemented with 10% FCS, 10 mM HEPES and 50 U/ml Penicillin-Streptomycin. On the next day, cells were transfected with 21 μg of the retroviral construct with Turbofect transfection reagent (Thermo Fischer Scientific), according to the manufacturer protocol. The medium was changed daily and collected at 48 h and 72 h post transfection. 48 h and 72 h virus supernatants were pooled and sedimented at 22000rcf for 2 h at 4° C. Finally, retrovirus pellets were resuspended in 2 ml of full RPMI medium and divided in 8 aliquots of 250 μl each, which were snap-frozen on dry ice and stored at −80° C.

For T cell transduction, a modified protocol from Tschumi et al, J Immunother Cancer. 2018 Jul. 13; 6(1):71 was used.

Spleens from wild type CD45.1.2 mice were smashed on a 70 m cell strainer. CD8 T cells were purified using the EasySep™ Mouse CD8+ T Cell Isolation Kit (StemCell) according to the manufacturer protocol. 0.5×10⁶ CD8 T cells were plated in 48 well plates in 0.5 ml of complete RPMI 1640 medium supplemented with 10% FCS, antibiotics and 50 IU/ml of recombinant human IL-2, and exposed to either DMSO or 20 μM UK5099. Mouse T-cells were activated with Activator CD3/CD28 Dynabeads (Gibco) at a ratio of 2 beads per cell. Retroviral infection was conducted at 37° C. for 24 h. Untreated 48-well plates were coated for 24 h with g/ml of recombinant human fibronectin (Takara Clontech) at 4° C., followed by PBS 2% BSA for 30 min at RT and finally washed with PBS. One aliquot of concentrated retroviruses was plated in each fibronectin-coated 48-well plates and centrifuged for 90 min at 2000rcf and 32° C. Then, 0.5×10⁶ of 24 h-activated CD8 T cells were added on top of the viruses and spun for 10 min at 400rcf and 32° C. On day 3, the medium was replaced with 10 IU/ml recombinant human IL-2, 10 ng/ml recombinant human IL-7 and 10 ng/ml recombinant human IL-15, containing either DMSO or 20 μM UK5099. Cells were then split every second day.

For adoptive cell transfer a modified protocol from Tschumi et al, J Immunother Cancer. 2018 Jul. 13; 6(1):71 was used.

CD45.2 C57BL/6 mice were engrafted subcutaneously with 4×10⁵ B16F10 tumors modified to express HER2. Six days later, mice were lymphodepleted with 100 mg/kg cyclophosphamide (Sigma Aldrich, C7397) injected i.p., and homogeneous groups were constituted with regard to tumor volume. T cells (5×10⁶) were adoptively transferred i.v. on the next day. Tumor volumes were measured three times a week with a caliper and calculated using the formula: V=π×[d2×D]/6, where d is the minor tumor axis and D is the major tumor axis. Tumors were collected and separated from skin. Single cell suspensions were obtained with the Mouse Tumor Dissociation Kit (Miltenyi, 130-096-730) according to the manufacturer protocol. Spleen and draining lymph node were smashed on a 70 m cell strainer. Single cell suspensions were stained with antibodies before flow cytometry analysis.

Polyclonal CD8 T cells from wild type mice were activated in the presence of DMSO or 20 μM UK5099 and then retrovirally transduced with a control blue fluorescent protein-expressing retroviral construct (BFP) or with HER2-CAR (FIG. 6A). When adoptively transferring (ACT) the T cells in mice bearing B16 melanoma tumors expressing HER2, we could observe that only UK5099-treated HER2CAR T cell ACT was able to significantly suppress tumor growth (FIG. 6B). More UK5099-HER2CAR T cells were engrafted in the tumor-draining lymph nodes, while CAR T cell numbers in the spleen were similar (FIGS. 6C and 6D). However, a higher percentage of splenic UK5099-HER2CAR T cells was expressing the memory-specific transcription factor TCF1 (FIG. 6E). Tumors of UK5099-HER2CAR-treated mice contained significantly more CAR T cells (FIG. 6F). As in the spleen, more tumor-infiltrating UK5099-treated CAR T cells expressed TCF1 (FIG. 6G). Interestingly, when looking at the co-expression of TCF1 with PD1, UK5099-treated CAR T cells formed more progenitor exhausted T cells (TCF1-positive) and less terminally differentiated exhausted T cells (TCF1-negative) (FIGS. 6H and 6I), indicative of an increased stem cell-like phenotype which might benefit from combination therapy with checkpoint blockade immunotherapy. Finally, fewer UK5099-treated CAR T cells were expressing the inhibitory molecules PD1 and TIM3, indicating a less exhausted phenotype (FIG. 6J).

### MPC Inhibition During the Activation and Culture of Adult Human T Cells Induces a Memory Phenotype.

MPC inhibition during activation of umbilical cord blood T cells induces a memory phenotype. This data demonstrates that these largely naive and stem-cell-like T cells can benefit from MPC inhibition, as those cord blood cells can have important applications as source cells for adoptive cell transfer therapies. However, the majority of the CAR T cell products currently approved or in clinical trials derive from patient (adult) T cells. It was thus intended to include new data showing the induction of memory phenotypes when adult T cells are activated and cultured with an MPC inhibitor (FIG. 7). As can be seen in that figure, not only the MPC inhibitor (UK5099) was used, but also the IDH2 inhibitor AG221 and a competitive molecule inhibiting PI3Kdelta (CAL101).

### Isolation and Culture of Peripheral Blood Mononuclear Cells (PBMCs)

Heparinised blood was diluted with PBS and pipetted on top of Ficoll-paque (LymphoPrep). After centrifugation at 1800 rpm for 20 minutes at room temperature, the layer of cells at the intersection, containing the PBMCs was removed, washed and resuspended at 5×10⁵ cells/ml of RPMI medium containing 8% human serum (AB serum), antibiotics, 2 mM L-Glutamine, 1% non-essential amino acids, 1 mM sodium pyruvate and 50 μM β-mercaptoethanol (all Gibco). Cells were activated with Human T-activator CD3/CD28 Dynabeads at a 1 bead/cell ratio with 150 IU/ml human recombinant IL2 and DMSO or 25 μM UK5099. Throughout the culture, cells were regularly split when necessary and beads were removed at day 5. At day 9, a fraction of the cells was stained with antibodies for flow cytometry analysis or for mitochondrial analysis.

### Mitochondrial Staining

2×10⁵ cells were stained with 25 nM TMRM and 50 nM MitoGreen (Molecular Probes, Invitrogen) for 30 minutes in RPMI with 5% FCS at 37° C. Cells were then washed, stained with a Live/Dead dye and antibodies before acquisition by flow cytometry.

The activation of PBMCs in vitro (FIG. 7A) resulted in a CD8 T cell population that was largely positive for the memory marker CD62L. Nevertheless, both UK5099- and AG221-treatment were able to further induce the population of CD8 T cells positive for CD62L (FIG. 2B). More strikingly, the mean fluorescent intensity of the CD62L signal in the CD62L-positive cells was increased upon UK5099 and AG221 treatment (FIG. 7C). This means that the CD62L-positive cells express more CD62L molecules on a per cell basis. When looking at several markers known in previous literature to identify stem cell-like memory T cells, it was found that this population of cells increased upon treatment with UK5099 (FIG. 7D). Intact mitochondrial membrane function is a known characteristic of long-lasting memory T cells. UK5099 treatment did not impact the total mitochondrial mass (FIG. 7E), but significantly increased mitochondrial membrane potential (FIG. 7F), resulting in more polarised mitochondria (FIG. 7G), which could be indicative of an improved mitochondrial respiratory capacity.

1. An in vitro cell culture method comprising a step of contacting T-cells with an inhibitor of the mitochondrial pyruvate carrier (MPC inhibitor).

2. The method of claim 1, wherein T-cells with a memory phenotype are generated and/or maintained.

3. The method of claims 1 or 2 comprising a further step of obtaining the T-cells from the culture, thereby producing a cell population comprising T-cells with a memory phenotype.

4. The method of any one of claims 1 to 3, wherein the T-cells are activated during culture.

5. The method of any one of claims 1 to 4, wherein the T-cells are expanded during culture, for example for 3 to 5 weeks.

6. The method of any one of claims 1 to 5, wherein the T-cells comprise CD8+ T-cells.

7. The method of any one of claims 1 to 6, wherein the T-cells are mammalian cells, preferably human cells.

8. The method of any one of claims 1 to 7, wherein the T-cells are human umbilical cord blood mononuclear cells (CBMC) and/or peripheral blood mononuclear cells (PBMC).

9. The method of any one of claims 1 to 8, wherein the T-cells are autologous cells.

10. The method of any one of claims 1 to 8, wherein the T-cells are allogeneic cells.

11. The method of any one of claims 1 to 10, wherein the T-cells are tumor-infiltrating T-cells and/or obtained from tumor-infiltrating T-cells.

12. The method of any one of claims 1 to 11, wherein the T-cells are tumor-draining lymph node cells and/or obtained from tumor-draining lymph nodes.

13. The method of any one of claims 1 to 12, wherein the T-cells comprise a heterologous antigen receptor, preferably a T-cell receptor (TCR) or a chimeric antigen receptor (CAR).

14. The method of any one of claims 1 to 13, wherein the T-cells are contacted with the MPC inhibitor from the beginning of the culture and/or activation.

15. The method of any one of claims 1 to 14, wherein the T-cells are contacted with the MPC inhibitor at least during activation.

16. The method of any one of claims 1 to 15, wherein the T-cells are contacted with the MPC inhibitor during the entire culture period.

17. The method of any one of claims 1 to 16, wherein the T-cells are activated by contacting them with an antigenic peptide, in particular in the presence of antigen-presenting cells, and/or artificial antigen presenting cells.

18. The method of any one of claims 1 to 17, wherein the T-cells are activated by contacting them with anti-CD3 and anti-CD28 antibodies, wherein said antibodies may be in solution, coupled to beads and/or coupled to artificial antigen presenting cells.

19. The method of any one of claims 1 to 18, wherein the T-cells are further contacted with IL-2.

20. The method of any one of claims 1 to 19, wherein the T-cells are contacted with (i) the MPC inhibitor, (ii) IL-2, and (iii) anti-CD3 and anti-CD28 antibodies and/or an antigenic peptide, in particular wherein the MPC inhibitor (i) is present in the culture medium, and IL-2 (ii) and/or the anti-CD3 and anti-CD28 antibodies and/or antigenic peptide (iii) are present in the culture medium and/or attached to the surface of antigen presenting cells and/or artificial antigen presenting cells.

21. The method of any one of claims 1 to 20, wherein the T-cells are first contacted with (i) the MPC inhibitor, (ii) IL-2, and (iii) anti-CD3 and anti-CD28 antibodies and/or an antigenic peptide, and then with IL-2 and IL-7.

22. The method of any one of claims 1 to 21, wherein the T-cells are cultured in a medium comprising (i) the MPC inhibitor, (ii) IL-2, and (iii) anti-CD3 and anti-CD28 antibodies and/or an antigenic peptide.

23. The method of any one of claims 1 to 22, wherein the T-cells are first cultured in a medium comprising (i) the MPC inhibitor, (ii) IL-2, and (iii) anti-CD3 and anti-CD28 antibodies and/or an antigenic peptide and then in a second medium comprising TL-2 and IL-7.

24. The method of any one of claims 1 to 23, wherein the MPC inhibitor comprises a small molecule, a nucleotide or a precursor thereof which interferes with Mpc1 and/or Mpc2 RNA (siRNA or shRNA), and/or an antibody and/or monobody.

25. The method of any one of claims 1 to 24, wherein the MPC inhibitor comprises at least one small molecule and/or an siRNA, preferably at least one small molecule.

26. The method of claim 25, wherein the at least one small molecule comprises UK5099 Pioglitazone, Rosiglitazone, MSDC-0602, MSDC-0160 and/or Zaprinast, in particular wherein UK5099 is 2-cyano-3-(1-phenyl-1H-indol-3-yl)-2-propenoic acid.

27. The method of any one of claims 1 to 26, wherein the MPC inhibitor comprises UK5099.

28. The method of claims 26 or 27, wherein the concentration of UK5099 is 25, 50 or 75 μM, preferably 25 μM.

29. A cell population comprising T-cells with a memory phenotype obtained by the method of any one of claims 3 to 28, preferably wherein the T-cells are human cells.

30. The cell population of claim 29 or the method of any one of claims 3 to 28, wherein at least 30%, 40%, 50%, 60%, 70%, 80%, or 90% of the cells in the population are T-cells with a memory phenotype, in particular wherein said T-cells with a memory phenotype express CD62L, preferably wherein said CD62L expression is at the cell surface.

31. The cell population of claims 29 or 30, wherein at least 90%, 95%, 98% or 99% of the cells in the population are T-cells.

32. The cell population of any one of claims 29 to 31, wherein at least 90%, 95%, 98% or 99% of the cells in the population are CD8+ T-cells.

33. The cell population of any one of claims 29 to 32 or the method of any one of claims 3 to 28, wherein the cell population comprises a higher proportion of T-cells with a memory phenotype and/or shows in average a more pronounced memory phenotype compared to a control cell population, in particular wherein the control cell population is obtained in parallel by the same method except that the control T-cells have not been contacted with an MPC inhibitor.

34. The cell population of any one of claims 29 to 33, or the method of any one of claims 3 to 28, wherein the T-cells maintain a memory phenotype in vivo when administered to a subject.

35. The cell population of any one of claims 29 to 34, or the method of any one of claims 3 to 28, wherein the T-cells efficiently give rise to memory precursor effector T-cells (MPECs) and/or central memory T-cells in vivo, for example in the spleen, when administered to a subject, in particular upon reencounter of the antigen.

36. The cell population of any one of claims 29 to 35, or the method of any one of claims 3 to 35, wherein the T-cells give rise to T-cells in vivo when administered to a subject, which have an unaltered or enhanced capacity of producing IFNγ and/or TNF upon restimulation, in particular upon reencounter of the antigen.

37. The cell population of the method of claim 36, wherein the T-cells are CD8+ T-cells that give rise to tumor-infiltrating cells in vivo when administered to the subject.

38. The cell population of any one of claims 29 to 37, or the method of any one of claims 3 to 28, wherein the T-cells give rise to T-cells, i.e. tumor-infiltrating cells, in vivo when administered to a subject, which have a decreased expression of PD-1.

39. The cell population of any one of claims 29 to 38, or the method of any one of claims 3 to 28, wherein the T-cells are CD8+ T-cells that give rise to memory precursor effector (MPEC) T-cells and/or central memory (CM) T-cells and/or tumor-infiltrating T-cells in vivo when administered to a subject, wherein said tumor-infiltrating T-cells have a decreased expression of PD-1 and/or an enhanced capacity of producing IFNγ and/or TNF upon reencounter of the antigen.

40. The cell population of any one of claims 29 to 39 or the method of any one of claims 1 to 28, wherein the T-cells are not contacted with an AKT inhibitor and/or have not been contacted with an AKT inhibitor.

41. The cell population of any one of claims 29 to 40 or the method of any one of claims 1 to 28, wherein the T-cells are not contacted with an MPC inhibitor and/or have not been contacted with an MPC inhibitor.

42. The cell population of any one of claims 29 to 41 or the method of any one of claims 1 to 28, wherein the T-cells are further contacted with an MPC inhibitor and/or have been contacted with an MPC inhibitor, preferably wherein said MPC inhibitor comprises AG221 and/or AGI6780, in particular wherein AG221 is 2-methyl-1-[[4-[6-(trifluoromethyl)pyridin-2-yl]-6-[[2-(trifluoromethyl)pyridin-4-yl]amino]-1,3,5-triazin-2-yl]amino]propan-2-ol and AGI6780 is 1-[5-(cyclopropylsulfamoyl)-2-thiophen-3-ylphenyl]-3-[3-(trifluoromethyl)phenyl]urea.

43. The cell population of any one of claims 29 to 42, or the method of any one of claims 3 to 28, wherein at least 40%, 50% 60%, 70%, 80%, or 90% are human central memory T-cells and/or human T-cells that co-express CD45RO and CCR7 and preferably CD62L, and preferably have no or low expression of CD45RA.

44. The cell population of any one of claims 29 to 43, wherein at least 40%, 50% 60%, 70%, 80%, or 90%, preferably at least 60%, of the cells in the population are human CD8+ T-cells with a memory phenotype that express CD62L, wherein said CD8+ T-cells have not been contacted with an AKT inhibitor.

45. The cell population of any one of claims 29 to 44, wherein said cell population is comprised in an in vitro cell culture.

46. The cell population of claim 45, wherein the cell culture comprises an MPC inhibitor.

47. The method of any one of claims 2 to 28 or the cell population of any one of claims 29 to 46, wherein the memory phenotype comprises expression of at least one memory marker selected from the group consisting of: CD62L (Sell), TCF1 (TCF7), CD27, CD127, CCR7 and CD28.

48. The method of any one of the preceding claims or the cell population of any one of claims 29 to 47, wherein the memory phenotype comprises absence of detectable expression of the non-memory marker KLRG1.

49. The method of any one of the preceding claims or the cell population of any one of claims 29 to 48, wherein the memory phenotype comprises expression of CD45RO, CCR7, CD27, CD28 and no or low expression of CD45RA, in particular wherein the T-cells with a memory phenotype are human cells.

50. The method of any one of the preceding claims or the cell population of any one of claims 29 to 49, wherein the memory phenotype comprises expression of the memory marker(s) CD62L and/or TCF1, in particular CD62L.

51. The method of any one of the preceding claims or the cell population of any one of claims 29 to 50, wherein the memory phenotype comprises surface expression of the memory marker CD62L.

52. The method of any one of any one of the preceding claims or the cell population of any one of claims 29 to 51, wherein the T-cells with a memory phenotype, i.e. the in vivo progeny thereof, (i) express CD62L and CD44 and/or (ii) express CD127 and lack KLRG1 expression, in particular wherein said cells in (i) are central memory T-cells, and the cells in (ii) are memory precursor effector T-cells.

53. The method of any one of the preceding claims or the cell population of any one of claims 29 to 52, wherein the memory phenotype comprises an increased basal oxygen consumption, maximal respiratory capacity and/or spare respiratory capacity, i.e. compared to the respective parameters in a control cell population, wherein the control T-cells have not been contacted with an MPC inhibitor.

54. The method of any one of the preceding claims or the cell population of any one of claims 29 to 53, wherein the memory phenotype comprises an open chromatin configuration, in particular wherein the open chromatin configuration is characterized by an increased trimethylation on the lysine 4 residue of histone 3 (H3K4-3Me), an increased acetylation on lysine 27 residue of histone 3 (H3K27-Ac) and/or more accessible chromatin regions, i.e. compared to the respective parameters in a control cell population, wherein the control T-cells have not been contacted with an MPC inhibitor.

55. The method of any one of the preceding claims or the cell population of any one of claims 29 to 54, wherein the memory phenotype comprises an open chromatin configuration at one or more, preferably at least 2, 3, 4 or 5, regulatory regions of at least one gene selected from the group consisting of: Sell (CD62L), Tcf7 (Tcf1), and Ccr7.

56. The method of any one of the preceding claims or the cell population of any one of claims 29 to 55, wherein the T-cells express at least one activation marker, in particular wherein said at least one activation marker is selected from the group consisting of: CD25, CD44, CD71 and CD98.

57. The method of any one of the preceding claims or the cell population of any one of claims 29 to 56, wherein the T-cells with a memory phenotype have a higher concentration of Acetyl-CoA.

58. The method of any one of the preceding claims or the cell population of any one of claims 29 to 57, wherein the T-cells with a memory phenotype incorporate carbon atoms from glutamine more efficiently into Acetyl-CoA than carbon atoms from glucose.

59. The method of any one of the preceding claims or the cell population of any one of claims 29 to 58, wherein more Acetyl-CoA in the T-cells with a memory phenotype is derived from glutamine than from glucose, in particular wherein more than 20%, 30%, 40%, 50% or 60% of the Acetyl-CoA is derived from glutamine and/or less than 20% of the Acetyl-CoA is derived from glucose.

60. The cell population of any one of claims 29 to 59 for use in immunotherapy, in particular wherein the cell population or the T-cells comprised in said cell population is/are administered to a subject.

61. An MPC inhibitor for use in immunotherapy.

62. The MPC inhibitor for use according to claim 61, wherein the immunotherapy comprises administering the MPC inhibitor to a subject.

63. The MPC inhibitor for use according to claims 61 or 62, wherein the immunotherapy comprises administering T-cells to a subject, i.e. by adoptive cell transfer, wherein said T-cells have been contacted with the MPC inhibitor during in vitro culture according to the method of any of the preceding claims, in particular wherein said T-cells have thereby acquired a memory phenotype in vitro.

64. The cell population for use according to claim 60 or the MPC inhibitor for use according to any one of claims 61 to 63, wherein the subject is a mammal, preferably a human, a domestic animal, or a pet, more preferably a human, most preferably a human patient in need for therapy.

65. The cell population or the MPC inhibitor for use according to the preceding claims, wherein the immunotherapy is a therapy for treating cancer, a chronic viral infection or an autoimmune disease.

66. The cell population or the MPC inhibitor for use according to claim 65, wherein the immunotherapy is a therapy for treating cancer, in particular an advanced cancer, preferably wherein the cancer, e.g the advanced cancer, is resistant to chemotherapy, therapy with an immune checkpoint inhibitor, targeted therapy and/or antibody-mediated immunotherapy and/or wherein the cancer comprises metastases.

67. The cell population or the MPC inhibitor for use according to claims 65 or 66, wherein the cancer is a hematological malignancy and/or a solid tumor, wherein said solid tumor is resistant to therapy with an immune checkpoint inhibitor (primary immune checkpoint blockade resistance) and/or acquires resistance to such a therapy;
wherein the chronic viral infection is HIV or SARS-CoV-2, preferably HIV; and/or
wherein the autoimmune disease is caused by and/or associated with autoreactive and/or pathogenic T-cells.

68. The cell population or the MPC inhibitor for use according to any one of the preceding claims, wherein the T-cells comprise CD8+ T-cells, wherein the T-cells are autologous cells, and/or wherein the T-cells are obtained from tumor-infiltrating T-cells.

69. The cell population or the MPC inhibitor for use according to any one of the preceding claims, wherein the T-cells comprise a heterologous antigen receptor, preferably a T-cell receptor (TCR) or a chimeric antigen receptor (CAR).

70. The cell population or the MPC inhibitor for use according to any one of the preceding claims, wherein an additional anti-cancer drug, preferably a checkpoint inhibitor, is administered to the patient.

71. An MPC inhibitor for use as a vaccine adjuvant.

72. A composition, in particular a pharmaceutical composition, comprising a vaccine and an MPC inhibitor.

73. The composition of claim 72, wherein said composition promotes the formation of T-cells with a memory phenotype in vivo when administered to a subject, in particular wherein said T-cells are activated.

74. The composition of claims 72 or 73, wherein said composition increases the number of T-cells expressing CD127 and having no or low expression of KLRG1, i.e. memory precursor effector T-cells, in vivo when administered to a subject compared to the administration of the respective vaccine without an MPC inhibitor.

75. A kit comprising (i) a vaccine and an MPC inhibitor, and/or (ii) the composition of any one of claims claim 72 to 74.

76. The MPC inhibitor for use according to claim 71, the composition of any one of claims 72 to 74 or the kit of claim 75, wherein the vaccine is a subunit vaccine.

77. The MPC inhibitor for use according to claims 71 or 76, the composition of any one of claims 72 to 74 or 76 or the kit of claims 75 or 76, wherein the vaccine comprises a further adjuvant such as an aluminium salt, AS01, AS04, MF59, a TLR agonist, and/or a STING agonist.

78. The MPC inhibitor for use according to any one of claims 71, 76 or 77, the composition of any one of claims 72 to 74, 76 or 77 or the kit of any one of claims 75 to 77, wherein the vaccine comprises an antigenic peptide, a nucleic acid encoding an antigenic peptide, a polysaccharide, a glycoprotein, a proteoglycane and/or a viral or bacterial vector comprising a nucleic acid encoding an antigenic peptide and/or the protein part of a glycoprotein and/or a proteoglycane, preferably wherein said vector encodes an antigenic peptide.

79. The MPC inhibitor for use according to any one of claims 71 or 76 to 78, the composition of any one of claims 72 to 74 or 76 to 78 or the kit of any one of claims 75 to 78, wherein the vaccine without an MPC inhibitor must be administered to a patient more than once to achieve the desired therapeutic and/or prophylactic effect, i.e. the required immunity.

80. The MPC inhibitor for use according to any one of claims 71 or 76 to 79, the composition of any one of claims 72 to 74 or 76 to 79 or the kit of any one of claims 75 to 79, wherein the vaccine is against cancer.

81. The MPC inhibitor for use according to any one of claims 71 or 76 to 80, the composition of any one of claims 72 to 74 or 76 to 80 or the kit of any one of claims 75 to 80, wherein the MPC inhibitor comprises at least one small molecule such as UK5099, Pioglitazone, Rosiglitazone, MSDC-0602, MSDC-0160 and/or Zaprinast, preferably at least 2-cyano-3-(1-phenyl-1H-indol-3-yl)-2-propenoic acid (UK5099).

82. The composition of any one of claims 72 to 74 or 76 to 81 or the kit of any one of claims 75 to 81 for use in treating and/or preventing a disease.

83. The composition or the kit for use according to claim 82, wherein the disease is cancer, or a chronic viral infection.

84. The composition or the kit for use according to claim 83, wherein the cancer is an advanced cancer, resistant to chemotherapy, therapy with an immune checkpoint inhibitor, targeted therapy and/or antibody-mediated immunotherapy and/or comprises metastases.

85. The composition or the kit for use according to claim 83 which is used for preventing the development of a preneoplastic lesion into a cancer, in particular wherein the preneoplastic lesion is Barretts's esophageous, cervical intraepithelial neoplasia, or a familial carcinoma such as familial melanoma, and/or characterized by germ line BRCA mutations associated with and/or leading to breast and ovarian carcinomas in women. In general, preneoplastic lesions refractory to standard of care and which invariable lead to tumor formation.

86. The composition or the kit for use according to claim 83 which is used for preventing a viral disease such as AIDS, or manifestation of a viral infection such as HIV.

87. The MPC inhibitor for use according to any one of claims 71 or 76 to 81, or the kit for use according to any one of claims 83 or 86, wherein the MPC inhibitor is administered to the subject prior to the vaccine, simultaneously with the vaccine, and/or subsequent to the vaccine, and/or more than once.

88. The MPC inhibitor for use according to any one of claims 71 or 76 to 81 or 87, or the kit for use according to any one of claims 83 to 87, wherein the MPC inhibitor is first administered to the patient together with the vaccine, and then at least once without the vaccine.
